(19) United States
(12) Patent Application Publication
(10) Pub. No.: US 2021/0221854 A1
(43) Pub. Date: Jul. 22, 2021

(54) SYSTEMS, METHODS, AND CELL LINES FOR GENERATING INFLUENZA VIRUS OR INFLUENZA VIRUS PROTEINS

(71) Applicant: Regents of the University of Minnesota, Minneapolis, MN (US)

(72) Inventors: Wei-Shou Hu, Minneapolis, MN (US); Thu Phan, Minneapolis, MN (US); Christopher S. Stach, Minneapolis, MN (US); Ryan A. Langlois, Minneapolis, MN (US); Qian Ye, Shanghai (CN)

(21) Appl. No.: 17/150,643

(22) Filed: Jan. 15, 2021

Related U.S. Application Data
(60) Provisional application No. 62/962,604, filed on Jan. 17, 2020.

(51) Int. Cl. C07K 14/11; C12N 15/87

(52) U.S. Cl. CPC C07K 14/11; C12N 2760/16023; C12N 15/87

(57) ABSTRACT

This disclosure describes a system for generating influenza virus or influenza virus proteins, methods of making that system, and methods of using that system.

### CONTINUING APPLICATION DATA

This application claims the benefit of U.S. Provisional Application Ser. No. 62/962,604, filed Jan. 17, 2020, which is incorporated by reference herein in its entirety.

### SEQUENCE LISTING

This application contains a Sequence Listing electronically submitted to the United States Patent and Trademark Office via EFS-Web as an ASCII text file entitled “0110-000638US01_ST25.txt” having a size of 11 kilobytes and created on Jan. 15, 2021. Due to the electronic filing of the Sequence Listing, the electronically submitted Sequence Listing serves as both the paper copy required by 37 CFR § 1.821(c) and the computer readable form (CRF) required by § 1.821(e). The information contained in the Sequence Listing is incorporated by reference herein.

### BACKGROUND

Influenza (flu) virus is responsible for seasonal flu epidemics each year. There are two main types of influenza virus, types A and B, that routinely spread in people. According to the Centers for Disease Control and Prevention (CDC), influenza has resulted in between 9 million and 45 million illnesses, between 140,000 and 810,000 hospitalizations, and between 12,000 and 61,000 deaths annually since 2010.

Influenza A viruses can be divided into subtypes based on two proteins on the surface of the virus: hemagglutinin (HA) and neuraminidase (NA). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Over the course of a flu season, different types and subtypes of influenza circulate and cause illness.

Influenza vaccination reduces the risk of flu illness by between 40% and 60% among the overall population, prevents tens of thousands of hospitalizations each year, and reduces the risks of the flu to children, pregnant women, older adults, and other at-risk populations.

Influenza vaccination is recommended yearly because a person's immune protection from vaccination declines over time and because flu vaccines are modified each season to protect against the viruses that research suggests may be most common during the upcoming flu season.

Current production of the flu vaccine is time-consuming and inefficient. Most flu vaccines include inactivated virus and are produced using a decades-old process that involves culturing viruses in hundreds of millions of embryonated chicken eggs. To acquire so many pathogen-free embryonated eggs is logistically challenging for the production of seasonally flu shots. In a pandemic situation when rapid ramping up of the production of flu vaccine is critical, an egg-based production faces even greater limitations. Additionally, the virus can mutate while it is growing in the eggs, resulting in a vaccine unable to block circulating subtypes.

### SUMMARY OF THE INVENTION

This disclosure describes a system for generating influenza virus or influenza virus proteins, methods of making that system, and methods of using that system. In contrast to the existing methods of producing influenza vaccine, the system described herein does not require the use of eggs, reduces or eliminates errors introduced by RNA-dependent genome replication, allows for controlled expression of components of the influenza virus, and allows for the rapid creation of cell lines producing different types and subtypes of influenza.

In one aspect, this disclosure describes a mammalian cell line that includes a first polynucleotide, a second polynucleotide, and a third polynucleotide that are stably integrated into the genome of the mammalian cell. The first polynucleotide encodes mRNA of a PB1 subunit of RNA-dependent RNA polymerase (RdRp) of influenza virus, and the first polynucleotide is operably linked to a first inducible promoter. The second polynucleotide encodes mRNA of a PB2 subunit of RdRp of influenza virus, and the second polynucleotide is operably linked to a second inducible promoter. The third polynucleotide encodes mRNA of a PA subunit of RdRp of influenza virus, and the third polynucleotide is operably linked to a third inducible promoter.

In some embodiments, the first polynucleotide, the second polynucleotide, and the third polynucleotide include DNA and encode positive-sense RNA.

In some embodiments, the mammalian cell line further includes one or more additional polynucleotides encoding mRNA of HA, NP, NA, M, or NS of influenza virus, or a combination thereof, wherein the one or more additional polynucleotides are stably integrated into the genome of the mammalian cell. The one or more additional polynucleotides encoding mRNA may be DNA.

In some embodiments, the mammalian cell line further includes one or more additional polynucleotides encoding vRNA of PB1, PB2, PA, HA, NP, NA, M, or NS of influenza virus, or a combination thereof, wherein the one or more additional polynucleotides are stably integrated into the genome of the mammalian cell. The one or more additional polynucleotides encoding vRNA may be DNA.

In another aspect, this disclosure describes transfecting the mammalian cell line with one or more additional polynucleotides. In some embodiments, the one or more additional polynucleotide may encode vRNA of PB1, PB2, PA, HA, NP, NA, M, or NS of influenza virus, or a combination thereof.

In a further aspect, this disclosure describes stably integrating in the mammalian cell line with one or more additional polynucleotides. In some embodiments, the one or more additional polynucleotide may encode vRNA of PB1, PB2, PA, HA, NP, NA, M, or NS of influenza virus, or a combination thereof.

In additional aspects, this disclosure describes methods of making the mammalian cell lines described herein.

In one aspect, this disclosure describes a method that includes stably integrating into a mammalian cell a first polynucleotide, a second polynucleotide, and a third polynucleotide that are stably integrated into the genome of the mammalian cell. The first polynucleotide encodes mRNA of a PB1 subunit of RNA-dependent RNA polymerase (RdRp) of influenza virus, and the first polynucleotide is operably linked to a first inducible promoter. The second polynucleotide encodes mRNA of a PB2 subunit of RdRp of influenza virus, and the second polynucleotide is operably linked to a second inducible promoter. The third polynucleotide encodes mRNA of a PA subunit of RdRp of influenza virus, and the third polynucleotide is operably linked to a third inducible promoter. The method may further include stably integrating a fourth polynucleotide, where the fourth polynucleotide encodes mRNA of influenza virus RNA-binding nucleoprotein (NP).

In some embodiments, the method further includes stably integrating into the mammalian cell one or more additional polynucleotides, the one or more additional polynucleotide encoding vRNA of PB1, PB2, PA, HA, NP, NA, M, or NS of influenza virus, or a combination thereof.

In another aspect, this disclosure describes a mammalian cell line that includes: a first polynucleotide encoding mRNA of a PB1 subunit of RNA-dependent RNA polymerase (RdRp) of influenza virus, wherein the first polynucleotide is operably linked to a first inducible promoter; a second polynucleotide encoding mRNA of a PB2 subunit of RdRp of influenza virus, wherein the second polynucleotide is operably linked to a second inducible promoter; a third polynucleotide encoding mRNA of a PA subunit of RdRp of influenza virus, wherein the third polynucleotide is operably linked to a third inducible promoter; a fourth polynucleotide encoding mRNA of influenza virus RNA-binding nucleoprotein (NP), wherein the fourth polynucleotide is operably linked to a fourth inducible promoter; a fifth polynucleotide encoding vRNA of influenza virus NP, wherein the fifth polynucleotide is operably linked to a first RNA polymerase promoter and a first RNA polymerase terminator; a sixth polynucleotide encoding vRNA of influenza virus non-structural protein (NS), wherein the sixth polynucleotide is operably linked to a second RNA polymerase promoter and a second RNA polymerase terminator; a seventh polynucleotide encoding vRNA of influenza virus matrix protein (M), wherein the seventh polynucleotide is operably linked to a third RNA polymerase promoter and a third RNA polymerase terminator; an eighth polynucleotide encoding vRNA of a PB1 subunit of RdRp of influenza, wherein the eighth polynucleotide is operably linked to a fourth RNA polymerase promoter and a fourth RNA polymerase terminator; a ninth polynucleotide encoding vRNA of a PB2 subunit of RdRp of influenza, wherein the ninth polynucleotide is operably linked to a fifth RNA polymerase promoter and a fifth RNA polymerase terminator; and a tenth polynucleotide encoding vRNA of a PA subunit of RdRp of influenza, wherein the tenth polynucleotide is operably linked to a sixth RNA polymerase promoter and a sixth RNA polymerase terminator.

In some embodiments, the mammalian cell line may further include an eleventh polynucleotide encoding vRNA of influenza virus neuraminidase (NA), wherein the eleventh polynucleotide is operably linked to a seventh RNA polymerase promoter and a seventh RNA polymerase terminator; and/or a twelfth polynucleotide encoding vRNA of influenza virus hemagglutinin (HA), wherein the twelfth polynucleotide is operably linked to an eighth RNA polymerase promoter and an eighth polymerase terminator.

In some embodiments, the mammalian cell line may further include an eleventh polynucleotide encoding an influenza virus genome segment wherein the eleventh polynucleotide is operably linked to a seventh RNA polymerase promoter and a seventh RNA polymerase terminator. In such embodiments, the influenza virus genome segment includes 3′ and 5′ packaging elements of vRNA of influenza virus hemagglutinin (HA), the influenza virus genome segment includes a coding sequence of influenza HA vRNA, and the influenza virus genome segment includes a defect in the coding sequence of influenza HA vRNA.

In some embodiments, the first polynucleotide of the mammalian cell line further includes the eighth polynucleotide; the second polynucleotide further includes the ninth polynucleotide; the third polynucleotide further includes the tenth polynucleotide; and a fourth polynucleotide further includes the fifth polynucleotide.

In some embodiments, the first polynucleotide, the second polynucleotide, the third polynucleotide, the fourth polynucleotide, the fifth polynucleotide, the sixth polynucleotide, the seventh polynucleotide, the eighth polynucleotide, the ninth polynucleotide, or the tenth polynucleotide, or any combination thereof are stably integrated into the genome of the mammalian cell line.

In yet another aspect, this disclosure describes methods of using the mammalian cell lines including, for example, to generate an infectious influenza virus particle or to generate an influenza virus protein. In some embodiments, the influenza virus particle is a replication-deficient influenza virus particle. In some embodiments, the method may include exposing the mammalian cell line to inducers of the inducible promoters.

As used herein, the term “operably linked” refers to direct or indirect covalent linking. Thus, two domains that are operably linked may be directly covalently coupled to one another. Conversely, the two operably linked domains may be connected by mutual covalent linking to an intervening moiety (for example, a flanking sequence). Two domains may be considered operably linked if, for example, they are separated by the third domain, with or without one or more intervening flanking sequences.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The patent or application file contains at least one drawing and photograph executed in color. Copies of this patent or patent application publication with color drawing(s) and photograph(s) will be provided by the Office upon request and payment of the necessary fee.

FIG. 1A shows a schematic of an exemplary influenza virion. FIG. 1B shows a schematic of influenza virus vRNAs, mRNAs of Influenza A Virus (IAV), and mRNAs of Influenza B Virus (IBV). FIG. 1C shows a simplified schematic of the influenza-virus life cycle. Green lines represent negative-sense RNA; orange lines represent positive-sense RNA. RNA-dependent RNA polymerase (RdRp); virus-encoded, short RNAs (svRNA); complimentary RNA (cRNA).

FIG. 2A-FIG. 2D show production of an inducible RNA-dependent RNA polymerase (iRdRP)-integrated cell line and show that production of RdRP by iRdRP cells is functional and tunable. FIG. 2A shows a scheme for producing iRdRP cells. FIG. 2B shows transfection of an iRdRP-integrated cell with a plasmid containing nucleotides designed to generate both mRNA and vRNA of NP and a Minigenome plasmid, as described in Example 1, to test for iRdRP cell functionality under induced and uninduced conditions. FIG. 2C shows imaging data indicating that the RdRP in the iRdRP-integrated cell line is functional and controllable. Cells were transfected with NP and minigenome, left panel shows uninduced cells, right panel shows induced cells. FIG. 2D shows qRT-PCR data indicating that transcript expression of the integrated RdRP genes varied upon different induction conditions.

FIG. 3A-FIG. 3B show single-cycle flu virus packaging using an iRdRP cell line, as further described in Example 2. FIG. 3A shows a schematic of the procedure of packaging single-cycle virus using iRdRP-integrated cell line. FIG. 3B shows single-cycle viral particles were packaged using iRdRP-integrated cell line. No viral particle released was detected when the cell line was un-induced (left panel). Release of many single-cycle viral particles was detected when the cell line was induced with 5 μg/mL doxycycline (right panel).

FIG. 4A-FIG. 4B show production of infectious flu virus by plasmid transfection of an iRdRP cell line. FIG. 4A shows a schematic of an exemplary procedure for packaging fully infectious viral particles using an iRdRP-integrated cell line. The step in the dashed box is used for the amplification of the viral particles released for the ease of detection. FIG. 4B shows the results of an HA assay that indicates the presence of infectious, replication-competent influenza viral particles after transfection of the iRdRP-integrated cell line as described in Example 3. Infectious viral particles were detected only when iRdRP-integrated cells were induced with 5 μg/mL of doxycycline. These results indicate the iRdRP cells can generate virus even when NP gene was replaced by NP gene from another virus strain.

FIG. 5A shows a schematic of the integration of nucleotide sequences that encode 8 vRNAs into cells that inducibly express RdRP and NP (iRdRp-NP-integrated cell) to create cells that produce flu virus (iFlu-producing cell), as further described in Example 4A. FIG. 5B shows a schematic of the integration of sequences that encode for 6 vRNAs and 2 mRNAs into the iRdRP-integrated cell line. The results iFlu-producing cells inducibly express RdRP and produce the necessary components to package virus with different HA and NA antigens. These cells may be used package different flu virus subtypes once upon transient transfection with plasmids encoding HA and NA of the corresponding subtypes, as further described in Example 4B.

FIG. 6A shows a schematic of a vector that expresses four vRNAs of Influenza A Virus (IAV) flanked by a transposase-specific inverted terminal repeat sequences (ITRs), referred to as “vector 2.2” or “2.2,” as further described in Example 5. The backbone of the vector is outside of the dashed line (from blasticidin S resistance gene (BlastR) to Uribo Left, which is one of the two ITRs); the insert is inside of the dashed line (from PB2 to NS). This 2.2 vector was used to integrate DNA sequences that encode four vRNAs (PB2, PB1, PA, NS) into 293T-iRdRP cells to make 293T-iRdRP 2.2 cells. FIG. 6B shows an exemplary RT-PCR result showing the expression of the four vRNAs from integrated sequences in 293T-iRdRP 2.2 cells. RNA from 293T-iRdRP cells integrated with 2.2 was harvested from a cell pool (denoted as “Pool”) as well as 293T-iRdRP 2.2 single-cell clones (denoted as “c1” and “c2”). Exemplary RT-PCR results shows integration and expression of PB2, PB1, PA, and NS vRNA in the 293T-iRdRP 2.2 cells. FIG. 6C shows a schematic of an exemplary experiment to produce virus from 293T-iRdRP 2.2 cells. 293T iRdRP 2.2 cells were transiently transfected with four plasmids, each including a sequence that encodes for viral vRNA under the control of a Pol I promoter and a Pol I terminator and viral mRNA under the control of a Pol II promoter and a Pol II terminator. The four plasmids are capable of expressing vRNAs and mRNAs of NP, M, HA, or NA (collectively denoted as “4 vmRNAs). The transfected cells were then overlayed with MDCK cells (step in dashed box) to amplify the signal in a hemagglutinin (HA) assay, allowing for detection of virus. As a positive control (denoted as “[+]”), 293T-iRdRP 2.2 cells were transiently transfected with vector 2.2 (which provides vRNA of PB2, PB1, PA, NS) and 4 other plasmids that encode vRNAs for M, NA, HA, and NP. 293T-iRdRP 2.2 cells were induced by 5 μg/mL doxycycline in both conditions. FIG. 6D shows exemplary results of the experiment described in FIG. 6C. The results demonstrate that 293T-iRdRP 2.2 cells can generated infectious virus upon transfection with 4 vmRNAs.

### DETAILED DESCRIPTION OF ILLUSTRATIVE EMBODIMENTS

This disclosure describes a system for generating influenza virus or influenza virus proteins including cell lines used to generate the virus or virus proteins, methods of making that system, and methods of using that system. In contrast to the existing methods of producing influenza vaccine, the system, methods, and cell lines described herein do not require the use of eggs, reduce or eliminate errors introduced by RNA-dependent genome replication, allow for controlled expression of components of the influenza virus, and allow for the rapid creation of cell lines producing different types and subtypes of influenza.

### Influenza

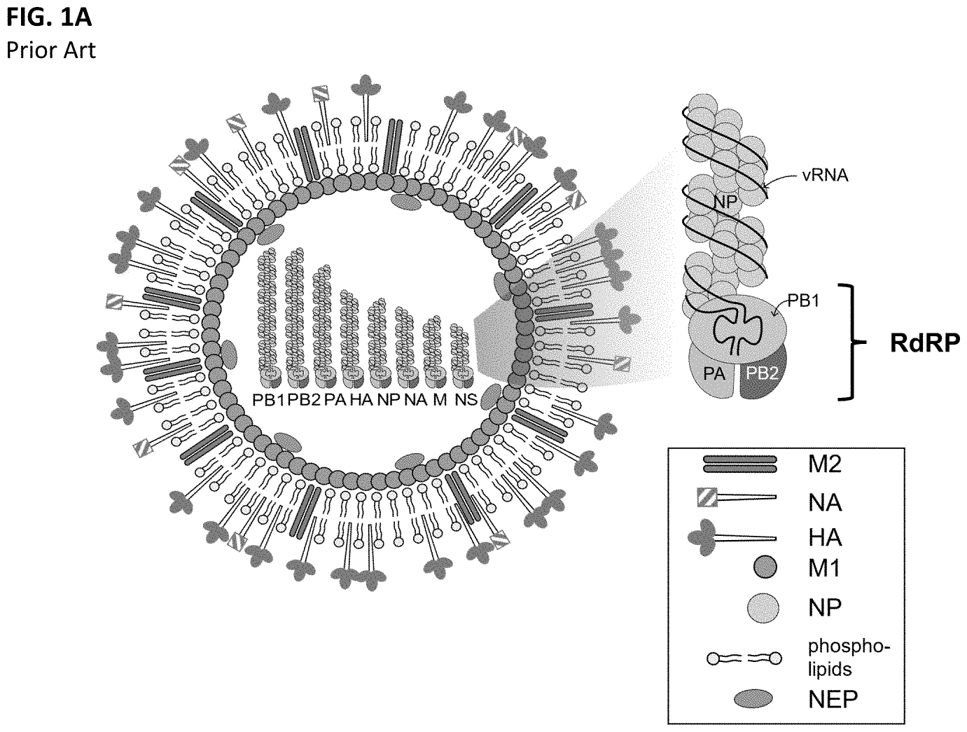

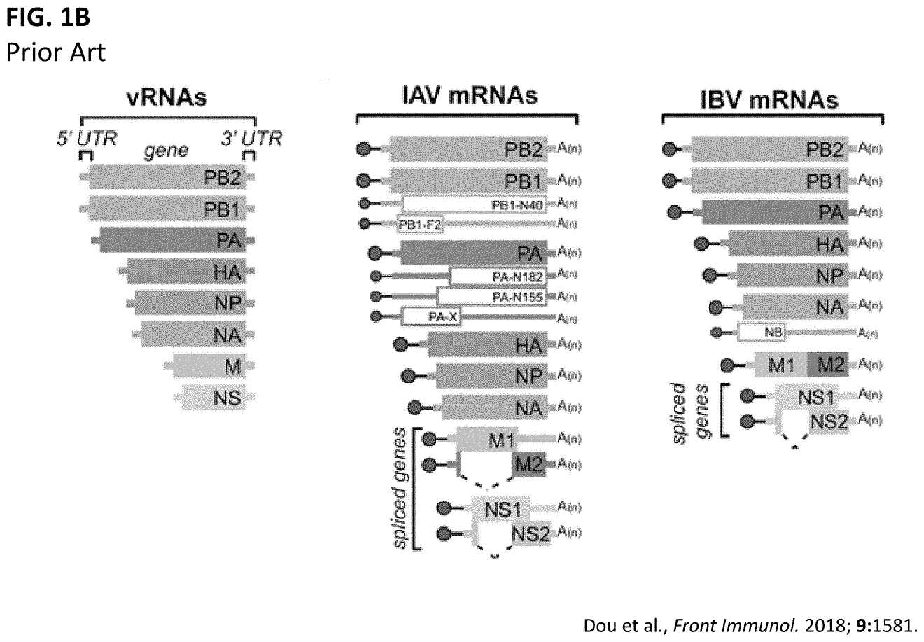

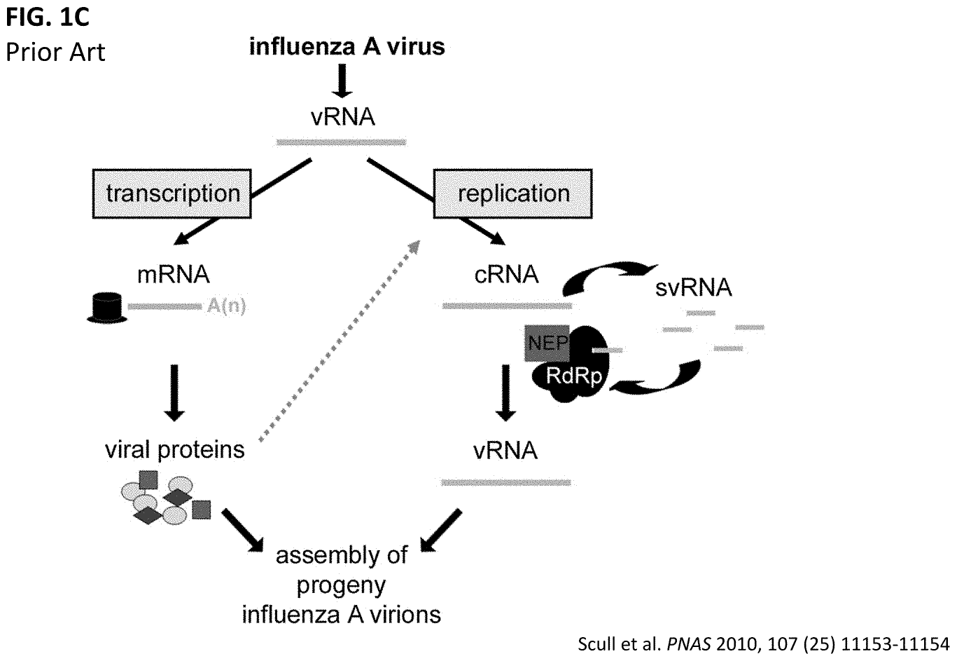

As shown in FIG. 1A, the influenza virion is a roughly spherical enveloped virus. The outer layer of the virion is a lipid membrane which is taken from the host cell in which the virus multiplies. Inserted into the lipid membrane are the glycoproteins (that is, protein linked to sugars) hemagglutinin (HA) and neuraminidase (NA). Variations in the HA and NA proteins determine the subtype of the virus including, for example, H5N1 or H1N1.

Also embedded in the lipid membrane is the M2 protein, a proton channel that traverses the viral envelope, equilibrating pH across the viral membrane during cell entry and across the trans-Golgi membrane of infected cells during viral maturation.

Inside the lipid membrane, the virus includes the viral protein M1 (or matrix protein); the nuclear export protein (NEP also referred to as NS2; the NS gene is spliced into non-structural protein NS1 and NEP or NS2); a single-strand RNA-binding nucleoprotein (NP); an RNA-dependent RNA polymerase (RdRp or RdRP), and eight viral RNAs (vRNAs).

M1 gives strength and rigidity to the lipid envelope.

NEP mediates the nuclear export of viral ribonucleoprotein (RNP) complexes, which are synthesized in the infected cell nucleus and are assembled into progeny virions at the cell membrane, and regulates the accumulation of viral genomic vRNA by the RdRP.

NP encapsidates the virus genome for the purposes of RNA transcription, replication and packaging. In the absence of NP, vRNAs are degraded.

RdRp transcribes and replicates influenza's negative-stranded RNA genome. Influenza virus's RdRp is a heterotrimeric complex of the proteins PB1, PB2, and PA.

As shown in FIG. 1B, the influenza virus genome is comprised of eight negative-sense, single-stranded viral RNA (vRNA) segments: NS, M, NA, NP, HA, PA, PB1, and PB2 (Dou et al. Front Immunol 9, 1581 (2018)). As shown in FIG. 1C, the vRNAs serve as templates for the transcription of viral mRNAs and complimentary RNAs (cRNAs) (Scull et al. Proc Natl Acad Sci USA 107, 11153-11154 (2010)). The mRNAs of Influenza A Virus (IAV) and Influenza B Virus (IBV) are shown in FIG. 1B.

As shown in FIG. 1C, cRNAs are replicated to produce more vRNAs. Influenza virus mRNA and cRNA/vRNA synthesis differ mechanistically. mRNA transcription is primed by capped RNA segments snatched from host cell pre-mRNAs by the viral polymerase. In contrast, cRNA/vRNA synthesis is primer-independent (Fan et al. Nature 573, 287-290 (2019)).

Each vRNA segment carries the coding sequence for one or two proteins in negative-sense orientation, flanked by 20- to 58-base untranslated regions (UTRs) at the 3′ and 5′ ends. Viral genomic packaging depends on the recognition of distinctive cis-acting packaging elements within the RNA. The cis packaging elements for influenza vRNA segments reside at both ends of each vRNA, including in the UTRs. Although up to 80 bases of adjacent coding sequences on any given segment may also be included in a packaging element (Liang et al. J Virol 82, 229-236 (2008)), the noncoding regions of the vRNA may be sufficient for the vRNA to be incorporated into a viral particle, while the coding regions typically serve as a bundling signal that ensures the incorporation of the complete set of eight vRNAs into the virion (Breen et al. Viruses 8, 179 (2016), Goto et al. J Virol 87, 11316-11322 (2013)).

### System for Generating Influenza Virus or Influenza Virus Proteins

In one aspect, this disclosure describes a system for generating influenza virus or influenza virus proteins.

In some embodiments, as further described below, the system includes a mammalian cell, wherein polynucleotides encoding mRNA of the subunits of RNA-dependent RNA polymerase (RdRp) are stably integrated into the genome of the mammalian cell.

In some embodiments, as further described below, the system includes a mammalian cell, wherein polynucleotides encoding mRNA of both the subunits of RdRp and NP are stably integrated into the genome of the mammalian cell.

In some embodiments, as further described herein, the mammalian cell may be transiently transfected with polynucleotides encoding a vRNA of influenza (for example a vRNA of PB1, PB2, PA, HA, NP, NA, M, or NS, or any combination thereof). Additionally, or alternatively, as further described herein, the mammalian cell may include polynucleotides encoding a vRNA of influenza (for example, a vRNA of PB1, PB2, PA, HA, NP, NA, M, or NS, or any combination thereof) which are stably integrated into the genome of the mammalian cell.

In some embodiments, the mammalian cell may be transiently transfected with polynucleotides encoding one or more additional mRNAs of influenza virus proteins (for example, HA, NP, NA, M, or NS, or a combination thereof). Additionally, or alternatively, as further described herein, the mammalian cell may include polynucleotides encoding one or more additional mRNAs of influenza virus proteins (for example, HA, NP, NA, M, or NS, or a combination thereof) which are stably integrated into the genome of the mammalian cell. As used herein, an mRNA of M or NS refers to an mRNA that provides both alternatively spliced mRNA products (M1 and M2 for M, and NS1 and NS2 for NS).

In some embodiments, the mammalian cell may be a cell of a mammalian cell line. Any suitable mammalian cell line may be used. For example, as shown in the Examples, the cell line may include a 293T cell. Additional exemplary cell lines may include a Madin-Darby Canine Kidney (MDCK) cell, a Vero cell, etc.

### RdRp-Integrated Mammalian Cell and RdRp-NP-Integrated Mammalian Cell

In one aspect, this disclosure describes a mammalian cell wherein polynucleotides encoding mRNA of the subunits of influenza virus RNA-dependent RNA polymerase (RdRp), PB1, PB2, and PA, are stably integrated into the genome of the mammalian cell. A mammalian cell line having PB1, PB2, and PA stably integrated its genome is referred to herein as an RdRp-integrated cell line. When PB1, PB2, and PA are operably linked to (and preferably under the control of) inducible promoters, as further described below, the mammalian cell line is referred to as an inducible RdRp (iRdRp)-integrated cell line.

The influenza virus may include any type of influenza virus (for example, Influenza A virus or Influenza B virus) or any subtype of influenza virus (for example, H1-H18 and/or N1-N11).

In yet another aspect, this disclosure describes a mammalian cell wherein polynucleotides encoding mRNA of PB1, PB2, and PA are stably integrated into the genome of the mammalian cell and a polynucleotide encoding mRNA of influenza virus RNA-binding nucleoprotein (NP) is stably integrated into the genome of the mammalian cell. A mammalian cell line having PB1, PB2, PA, and NP stably integrated its genome is referred to herein as an RdRp-NP-integrated cell line. When PB1, PB2, PA, and NP are operably linked to inducible promoters, as further described below, the mammalian cell line is referred to as an inducible RdRp-NP (iRdRp-NP)-integrated cell line.

In some embodiments, each subunit of RdRP is preferably under the control of an inducible promoter. The subunits may be under the control of the same inducible promoter. For example, as described in Example 1, each of the subunits may be under the control of the TetOn promoter. In some embodiments, however, the subunits may be under the control of different inducible promoters. For example, a first polynucleotide encoding mRNA of PB1 may be operably linked to a first inducible promoter, a second polynucleotide encoding mRNA of PB2 may be operably linked to a second inducible promoter, and a third polynucleotide encoding mRNA of PA may be operably linked to a third inducible promoter.

Any suitable inducible promoter may be used. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR.

In some embodiments, the inducible protomers are preferably RNA Polymerase II (Pol II) promoters. In some embodiments, polynucleotides encoding mRNA of PB1, PB2, and PA may also be operably linked to an RNA polymerase terminator. In some embodiments, the RNA polymerase terminator corresponds to the type (for example, Pol I, Pol II, or Pol III) of RNA polymerase promoter. In some embodiments, the RNA polymerase terminator preferably includes a Pol II terminator.

In some embodiments, including when a polynucleotide encoding mRNA of NP is stably integrated into the genome of the mammalian cell, NP is preferably under the control of an inducible promoter. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR. In some embodiments, NP is preferably under the control of a different inducible promoter than the inducible promoter or promoters used for the polynucleotide encoding the subunits of RdRp.

Placing polynucleotides encoding RdRP or encoding RdRP and NP under the control of one or more inducible promoters allows for the control of the levels of PB1, PB2, PA, and NP. When PB1, PB2, PA, and NP are under the control of different inducible promoters, their levels may be separately controlled. Moreover, because vRNAs are degraded in the absence of RdRP and NP, placing polynucleotides encoding RdRP and NP under the control of an inducible promoter also allows for the control of the levels of influenza vRNA in a cell. Additionally, having PB1, PB2, PA, and NP under the control of inducible promoters may help to limit the toxic effects of some viral genes or viral proteins on the cell (including, for example, the subunits of RdRP).

In some embodiments, a polynucleotide encoding mRNA of PB1, PB2, and PA encodes positive-sense RNA. In some embodiments, a polynucleotide encoding mRNA of NP encodes positive-sense RNA. By integrating the viral mRNA as DNA, the mutation frequency of the viral RNA is dramatically reduced.

In some embodiments, the RdRp-integrated cell may be transiently transfected with a polynucleotide that includes the mRNA of an influenza virus protein. For example, the RdRp-integrated cell may be transiently transfected with a polynucleotide that includes the mRNA of NP, HA, NA, M, or NS, or any combination thereof.

In an exemplary embodiment, the RdRp-integrated cell may be transiently transfected with a polynucleotide that includes the mRNA of influenza virus RNA-binding nucleoprotein (NP). For example, an iRdRp-integrated cell may be transiently transfected with a plasmid that includes the protein coding sequence of NP. While described in Example 1 in the context of an exemplary embodiment in which the mRNA of NP is introduced into an iRdRp-integrated cell by a plasmid and its expression is under the control of a Pol II promoter and a Pol II terminator, the mRNA of NP (or any other influenza protein) may be transiently introduced by any suitable method and using any suitable promoter or terminator or both.

In some embodiments, and as further described below, the RdRp-integrated cell may be transiently transfected with a polynucleotide that includes the mRNA of influenza virus protein and/or the vRNA of an influenza virus protein. In some embodiments, the polynucleotide may include the mRNA and the vRNA of the same influenza virus protein.

In an exemplary embodiment, the RdRp-integrated cell may be transiently transfected with a polynucleotide that includes the mRNA and the vRNA of influenza virus RNA-binding nucleoprotein (NP). For example, in Example 2, mRNA and vRNA for NP was introduced into an iRdRp-integrated cell by a plasmid and the vRNA expression was under the control of a Pol I promoter and a Pol I terminator and the mRNA expression is under the control of a Pol II promoter and a Pol II terminator. In another example, in Example 5, mRNA and vRNA for NP, M, HA, and NA (with each vRNA under the control of a Pol I promoter and a Pol I terminator and each mRNA under the control of a Pol II promoter and a Pol II terminator) was introduced into an iRdRp-integrated cell by 4 separate plasmids.

In some embodiments, the influenza virus genome segment is operably linked to an RNA polymerase promoter. The RNA polymerase promoter may include a Pol I, a Pol II, or a Pol III promoter. In some embodiments, the RNA polymerase is preferably a Pol I promoter; Pol I transcribes genes encoding ribosomal rRNA, facilitating the production of vRNA from a DNA sequence encoding the vRNA. In some embodiments where the RNA polymerase promoter includes a Pol II promoter, the influenza virus genome segment may be operably linked to ribozymes as described in U.S. Pat. No. 7,723,094. In some embodiments, the RNA polymerase promoter includes a human or a mouse promoter. In an exemplary embodiment, the RNA polymerase promoter includes a human Pol I promoter.

In some embodiments the influenza virus genome segment may be operably linked to an RNA polymerase terminator. In some embodiments, the RNA polymerase terminator corresponds to the type (Pol I, Pol II, or Pol III) of RNA polymerase promoter. In some embodiments, the RNA polymerase terminator preferably includes a Pol I terminator. In some embodiments, the RNA polymerase terminator includes a human or a mouse terminator. In an exemplary embodiment, the RNA polymerase terminator includes a mouse Pol I terminator.

In some embodiments, the mammalian cell may be transiently transfected with a polynucleotide encoding influenza virus vRNA. For example, the mammalian cell may be transiently transfected with plasmid including a polynucleotide encoding influenza virus vRNA. The influenza virus vRNA may include NS vRNA, M vRNA, NA vRNA, NP vRNA, HA vRNA, PA vRNA, PB1 vRNA, or PB2 vRNA, or any combination thereof. In some embodiments, the polynucleotide encoding the influenza virus vRNA may be operably linked to an RNA polymerase promoter or an RNA polymerase terminator or both. In some embodiments, the RNA polymerase terminator corresponds to the type (Pol I, Pol II, or Pol III) of RNA polymerase promoter. In some embodiments, the RNA polymerase promoter preferably includes a Pol I promoter. In some embodiments, the RNA polymerase terminator preferably includes a Pol I terminator. In some embodiments, the RNA polymerase promoter includes a human or a mouse promoter. In some embodiments, the RNA polymerase terminator includes a human or a mouse terminator.

In some embodiments, the mammalian cell may be transiently transfected with a plasmid comprising an influenza virus genome segment that includes the 3′ and 5′ untranslated regions (UTRs) of an influenza virus vRNA.

In some embodiments, when expression of HA and NA are desired, it may be preferable to transiently transfect an RdRp-integrated cell (including, for example, an iRdRp-integrated cell) with vRNA and mRNA HA and/or NA instead of stably integrating HA and/or NA into the genome of the mammalian cell. Because HA and NA control the subtype of the influenza virus, which changes seasonably, transiently transfecting HA and NA instead of stably integrating HA and NA may provide greater flexibility. An exemplary embodiment of such a cell is described in Example 4B.

Any suitable reporter gene may be included. In some embodiments, the reporter gene may be a fluorescence gene including, for example, GFP, RFP, etc. In some embodiments, the reporter gene may include a luciferase enzyme, β-galactosidase, or chloramphenicol acetyltransferase.

In some embodiments, the influenza virus genome segment includes a reporter gene that replaces at least a portion of the coding sequence of the influenza virus vRNA while retaining the cis packaging elements for the influenza virus vRNA. Inclusion of the cis packaging elements for the vRNA may be used to promote packaging of the reporter gene. In some embodiments, the packaging elements include the 5′ and 3′ untranslated regions of the vRNA. In some embodiments, the packaging elements include the 5′ and 3′ untranslated regions of the vRNA and an additional bundling signal in the coding sequence of the vRNA.

In an additional embodiment, the influenza virus vRNA may include a mutation or multiple mutations (for example, having a synergistic effect) in at least one of the polynucleotides encoding vRNA of the subunits of RdRp (PB1, PB2, and PA). In some embodiments, the mutation may introduce an inducible defect. For example, the defect could be heat-inducible. By introducing a mutation or defect in the vRNA of one or more of the subunits of RdRp, an infectious but single cycle viral particle may be generated.

### Influenza Virus (IV)-Integrated Mammalian Cell

In a further aspect, this disclosure describes a mammalian cell that includes polynucleotides encoding mRNAs of each influenza virus protein and vRNAs of each vRNA segment (NS, M, NA, NP, HA, PA, PB1, and PB2). The polynucleotides may be stably integrated and/or may be transiently transfected. At least some of the polynucleotides are stably integrated. In some embodiments, all of the polynucleotides are stably integrated. As further described herein, in some embodiments, it may be advantageous to transiently transfect HA and NA instead of stably integrating HA and NA.

In some embodiments, at least the polynucleotides encoding mRNAs of PA, PB1, and PB2 are stably integrated into the genome of the mammalian cell. In some embodiments, at least the polynucleotides encoding mRNAs of PA, PB1, PB2, and NP are stably integrated into the genome of the mammalian cell. Moreover, integrating polynucleotides encoding mRNAs for RdRp subunits under the control of inducible promoters or polynucleotides encoding mRNAs for both RdRp subunits and NP under the control of inducible promoters may allow for control of the dynamics of the induction and packaging of the virus. Such control may be achieved, for example, by the order or timing in which the inducible promoters are introduced to the cells. Moreover, because continuous expression of RdRp protein is toxic to cells, an inducible RdRp (iRdRp)-integrated cell line may be advantageous because it exhibits stable integration of RdRP while maintaining normal cell growth over time.

In some embodiments, the polynucleotides encoding mRNAs of PA, PB1, PB2, NP, M, and NS are stably integrated into the genome of the mammalian cell. Not integrating polynucleotides encoding mRNA of HA and NA may, in some embodiments, be advantageous because HA and NA control the subtype of the influenza virus, which changes seasonably. Thus, transiently transfecting HA and NA instead of stably integrating HA and NA may provide greater flexibility. In some embodiments, the polynucleotides encoding mRNAs of PA, PB1, PB2, HA, NP, NA, M, and NS are stably integrated into the genome of the mammalian cell.

In some embodiments, at least some of the polynucleotides encoding vRNA of each vRNA segment may be stably integrated into the genome of the mammalian cell. Any combination of polynucleotides encoding vRNA may also be integrated into the genome of the mammalian cell. For example, in some embodiments, vRNA of NS, PA, PB1, and PB2 may be stably integrated into the genome of the mammalian cell. In an exemplary embodiment, shown in Example 5, polynucleotides encoding vRNA of PA, PB1, PB2, and NS are stably integrated into the genome of the mammalian cell. In some embodiments, vRNA of NS, M, NP, PA, PB1, and PB2 may be stably integrated into the genome of the mammalian cell. Not integrating vRNA of HA and NA may, in some embodiments, be advantageous because HA and NA control the subtype of the influenza virus, which changes seasonably. Thus, transiently transfecting HA and NA instead of stably integrating HA and NA may provide greater flexibility. In some embodiments, vRNA of each vRNA segment (NS, M, NA, NP, HA, PA, PB1, and PB2) may be stably integrated into the mammalian cell.

In some embodiments, polynucleotides encoding an mRNA of an influenza virus protein and a vRNA of an influenza virus protein may be stably integrated into the genome of the mammalian cell. In some embodiments, the polynucleotide may include the mRNA and the vRNA of the same influenza virus protein.

In some embodiments, each polynucleotide (that is, polynucleotides encoding mRNAs of each influenza virus protein and vRNA of each vRNA segment) may be stably integrated into the genome of the mammalian cell. Such a mammalian cell would allow for the production of influenza virus without virus infection or multiple plasmid transfection.

As further described below, in some embodiments, the mammalian cell may include polynucleotides encoding functional mRNAs of each influenza virus protein and vRNA of each vRNA segment resulting in a mammalian cell that produces a replication competent influenza virus.

Alternatively, as further described below, the mammalian cell may include a defect in one or more of the polynucleotides encoding a vRNA, resulting in a mammalian cell that produces a replication-deficient influenza virus particle including, for example, a single-cycle influenza virus particle.

### Replication Competent Influenza Virus (IV)-Integrated Mammalian Cell

In some embodiments, a mammalian cell that produces a replication competent influenza virus may include a separate polynucleotide for each mRNA of influenza and each vRNA of influenza included in the construct, and each polynucleotide may be under separate promoter control.

For example, in an exemplary embodiment, a mammalian cell may include: a first polynucleotide encoding mRNA of a PB1 subunit of RdRp, wherein the first polynucleotide is operably linked to a first inducible promoter; a second polynucleotide encoding mRNA of a PB2 subunit of RdRp, wherein the second polynucleotide is operably linked to a second inducible promoter; a third polynucleotide encoding mRNA of a PA subunit of RdRp, wherein the third polynucleotide is operably linked to a third inducible promoter; a fourth polynucleotide encoding mRNA of NP, wherein the fourth polynucleotide is operably linked to a fourth inducible promoter. The mammalian cell may further include a fifth polynucleotide encoding vRNA of virus NP, wherein the fifth polynucleotide is operably linked to a first RNA polymerase promoter and a first RNA polymerase terminator; a sixth polynucleotide encoding vRNA of NS, wherein the sixth polynucleotide is operably linked to a second RNA polymerase promoter and a RNA polymerase terminator; a seventh polynucleotide encoding vRNA of HA, wherein the seventh polynucleotide is operably linked to a third RNA polymerase promoter and a third RNA polymerase terminator; an eighth polynucleotide encoding vRNA of M, wherein the eighth polynucleotide is operably linked to a fourth RNA polymerase promoter and a fourth RNA polymerase terminator; a ninth polynucleotide encoding vRNA of NA, wherein the polynucleotide is operably linked to a fifth RNA polymerase promoter and a fifth RNA polymerase terminator; a tenth polynucleotide encoding vRNA of a PB1 subunit of RdRp, wherein the tenth polynucleotide is operably linked to a sixth RNA polymerase promoter and a sixth RNA polymerase terminator; a eleventh polynucleotide encoding vRNA of a PB2 subunit of RdRp, wherein the eleventh polynucleotide is operably linked to a seventh RNA polymerase promoter and a seventh RNA polymerase terminator; and a twelfth polynucleotide encoding vRNA of a PA subunit of RdRp, wherein the twelfth polynucleotide is operably linked to an eighth RNA polymerase promoter and an eighth RNA polymerase terminator. In some embodiments, each of the polynucleotides may be stably integrated into the genome of the mammalian cell.

In another exemplary embodiment, a mammalian cell may include: a first polynucleotide encoding mRNA of a PB1 subunit of RdRp, wherein the first polynucleotide is operably linked to a first inducible promoter; a second polynucleotide encoding mRNA of a PB2 subunit of RdRp, wherein the second polynucleotide is operably linked to a second inducible promoter; a third polynucleotide encoding mRNA of a PA subunit of RdRp, wherein the third polynucleotide is operably linked to a third inducible promoter; a fourth polynucleotide encoding mRNA of NP, wherein the fourth polynucleotide is operably linked to a fourth inducible promoter. The mammalian cell may further include a fifth polynucleotide encoding vRNA of virus NP, wherein the fifth polynucleotide is operably linked to a first RNA polymerase promoter and a first RNA polymerase terminator; a sixth polynucleotide encoding vRNA of influenza virus non-structural protein (NS), wherein the sixth polynucleotide is operably linked to a second RNA polymerase promoter and a second RNA polymerase terminator; a seventh polynucleotide encoding vRNA of influenza virus matrix protein (M), wherein the seventh polynucleotide is operably linked to a third RNA polymerase promoter and a third RNA polymerase terminator; an eighth polynucleotide encoding vRNA of a PB1 subunit of RdRp of influenza, wherein the eighth polynucleotide is operably linked to a fourth RNA polymerase promoter and a fourth RNA polymerase terminator; a ninth polynucleotide encoding vRNA of a PB2 subunit of RdRp of influenza, wherein the ninth polynucleotide is operably linked to a fifth RNA polymerase promoter and a fifth RNA polymerase terminator; and a tenth polynucleotide encoding vRNA of a PA subunit of RdRp of influenza, wherein the tenth polynucleotide is operably linked to a sixth RNA polymerase promoter and a sixth RNA polymerase terminator. In some embodiments, each of the polynucleotides may be stably integrated into the genome of the mammalian cell.

In some embodiments, the mammalian cell line may further include an eleventh polynucleotide encoding vRNA of influenza virus neuraminidase (NA), wherein the eleventh polynucleotide is operably linked to a seventh RNA polymerase promoter and a seventh RNA polymerase terminator; and/or a twelfth polynucleotide encoding vRNA of influenza virus hemagglutinin (HA), wherein the twelfth polynucleotide is operably linked to an eighth RNA polymerase promoter and an eighth polymerase terminator. In some embodiments, the eleventh polynucleotide and/or twelfth polynucleotides may be stably integrated into the genome of the mammalian cell.

In some embodiments, one or more of the first inducible promoter, the second inducible promoter, and the third inducible promoter, and the fourth inducible promoter comprise different promoters. In some embodiments, each inducible promoter is a Pol II promoter. In some embodiments, a polynucleotide that is operably linked to one of the inducible promoters may also be operably linked to an RNA polymerase terminator. In some embodiments, the RNA polymerase terminator corresponds to the type (Pol I, Pol II, or Pol III) of the inducible promoter. In some embodiments, the RNA polymerase terminator preferably includes a Pol II terminator.

Any suitable inducible promoter or combination of promoters may be used. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR.

The RNA polymerase promoters and RNA polymerase terminators for the polynucleotides encoding vRNA are further discussed below.

In other embodiments, a mammalian cell that produces a replication competent influenza virus may include a single polynucleotide for the mRNA and vRNA of PB1, PB2, PA, and NP, wherein each of those polynucleotides includes two separate promoters—one for the mRNA and one for the vRNA. In some embodiments, the promoter for polynucleotide encoding the mRNA may be a Pol II promoter and the promoter for polynucleotide encoding the vRNA may be a Pol I promoter. The mammalian cell may further include additional polynucleotides for the vRNAs of the remaining influenza vRNAs.

In an exemplary embodiment, a mammalian cell may include: a first polynucleotide encoding mRNA and vRNA of a PB1 subunit of RdRp, wherein the first polynucleotide is operably linked to a first promoter and a second promoter; a second polynucleotide encoding mRNA and vRNA of a PB2 subunit of RdRp, wherein the second polynucleotide is operably linked to a third promoter and a fourth promoter; a third polynucleotide encoding mRNA and vRNA of a PA subunit of RdRp, wherein the third polynucleotide is operably linked to a fifth promoter and a sixth promoter; a fourth polynucleotide encoding mRNA and vRNA of influenza virus RNA-binding nucleoprotein (NP), wherein the fourth polynucleotide is operably linked to a seventh promoter and an eighth promoter; a fifth polynucleotide encoding vRNA of NS, wherein the fifth polynucleotide is operably linked to a ninth promoter; a sixth polynucleotide encoding vRNA of HA, wherein the sixth polynucleotide is operably linked to a tenth promoter; a seventh polynucleotide encoding vRNA of M, wherein the seventh polynucleotide is operably linked to a eleventh promoter; and an eighth polynucleotide encoding vRNA of NA, wherein the polynucleotide is operably linked to an twelfth promoter.

In some embodiments, one or more of the promoters are inducible promoters. In some embodiments, one or more of the polynucleotides encoding an mRNA is operably linked to an inducible promoter. In some embodiments, each of the polynucleotides encoding a mRNA is operably linked to an inducible promoter. In some embodiments, the inducible promoters are preferably RNA Pol II promoters. In some embodiments, a polynucleotide that is operably linked to an inducible promoter (including, for example, an RNA Pol II promoter) is also operably linked to an RNA polymerase terminator. In some embodiments, the RNA polymerase terminator corresponds to the type (for example, Pol I, Pol II, or Pol III) of the inducible promoter. In some embodiments, the RNA polymerase terminator preferably includes a Pol II terminator.

Any suitable inducible promoter or combination of promoters may be used. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR.

In some embodiments, one or more of the polynucleotides encoding an vRNA is operably linked to an RNA polymerase promoter. In some embodiments, each of the polynucleotides encoding an vRNA is operably linked to an RNA polymerase promoter.

An RNA polymerase promoter may include any suitable RNA polymerase promoter. In some embodiments, the RNA polymerase promoter for a polynucleotide encoding a vRNA preferably includes a Pol I promoter. Pol I transcribes genes encoding ribosomal rRNA, facilitating the production of vRNA from a DNA sequence encoding the vRNA. In some embodiments, where the RNA polymerase promoter includes a Pol II promoter, the influenza virus genome segment may be operably linked to ribozymes as described in U.S. Pat. No. 7,723,094. In some embodiments, the RNA polymerase promoter includes a human or a mouse promoter. In an exemplary embodiment, the RNA polymerase promoter for a polynucleotide encoding a vRNA includes a human Pol I promoter.

In some embodiments a polynucleotide encoding an vRNA is also operably linked to an RNA polymerase terminator. An RNA polymerase terminator may include any suitable RNA polymerase terminator. In some embodiments, the RNA polymerase terminator corresponds to the type (Pol I, Pol II, or Pol III) of an RNA polymerase promoter. In some embodiments, the RNA polymerase terminator for a polynucleotide encoding a vRNA preferably includes a Pol I terminator. In some embodiments, the RNA polymerase terminator includes a human or a mouse terminator. In an exemplary embodiment, the RNA polymerase terminator for a polynucleotide encoding a vRNA includes a mouse Pol I terminator.

### Single-Cycle Influenza Virus (IV)-Integrated Mammalian Cell

In another aspect, this disclosure describes a mammalian cell that produces a replication-deficient influenza virus particle including, for example, a single-cycle influenza virus particle. As noted above, if the mammalian cell includes a defect in one or more of the polynucleotides encoding a vRNA, a mammalian cell that produces a single-cycle (that is, replication incompetent) influenza virus may result.

In some embodiments a mammalian cell that produces a single-cycle influenza virus may include a separate polynucleotide for each mRNA of influenza virus and each vRNA of influenza virus included in the construct, and each polynucleotide may be under separate promoter control. To achieve a single-cycle influenza virus, the coding sequence of at least one vRNA may preferably be defective, resulting in a virus may be packaged only if a polynucleotide encoding the protein that would otherwise be encoded by the gene is supplied.

In some embodiments described herein, the coding sequence of HA vRNA is defective, resulting in a virus may be packaged only if a polynucleotide encoding HA is otherwise supplied. This example is not meant to be limiting, however. Embodiments in which the coding sequence of a different vRNA is defective are also envisioned.

For example, in an exemplary embodiment, a mammalian cell may include: a first polynucleotide encoding mRNA of a PB1 subunit of RdRp, wherein the first polynucleotide is operably linked to a first inducible promoter; a second polynucleotide encoding mRNA of a PB2 subunit of RdRp, wherein the second polynucleotide is operably linked to a second inducible promoter; a third polynucleotide encoding mRNA of a PA subunit of RdRp, wherein the third polynucleotide is operably linked to a third inducible promoter; a fourth polynucleotide encoding mRNA of NP, wherein the fourth polynucleotide is operably linked to a fourth inducible promoter; a fifth polynucleotide encoding vRNA of NP, wherein the fifth polynucleotide is operably linked to a first RNA polymerase promoter and a first RNA polymerase terminator; a sixth polynucleotide encoding vRNA of NS, wherein the sixth polynucleotide is operably linked to a second RNA polymerase promoter and a second RNA polymerase terminator; a seventh polynucleotide encoding vRNA of M, wherein the seventh polynucleotide is operably linked to a third RNA polymerase promoter and a third RNA polymerase terminator; an eighth polynucleotide encoding vRNA of NA, wherein the eighth polynucleotide is operably linked to a fourth RNA polymerase promoter and a fourth RNA polymerase terminator; a ninth polynucleotide encoding vRNA of a PB1 subunit of RdRp, wherein the ninth polynucleotide is operably linked to a fifth RNA polymerase promoter and a fifth RNA polymerase terminator; a tenth polynucleotide encoding vRNA of a PB2 subunit of RdRp, wherein the tenth polynucleotide is operably linked to a sixth RNA polymerase promoter and a sixth RNA polymerase terminator; a eleventh polynucleotide encoding vRNA of a PA subunit of RdRp, wherein the eleventh polynucleotide is operably linked to a seventh RNA polymerase promoter and a seventh RNA polymerase terminator; and a twelfth polynucleotide encoding an influenza virus genome segment wherein the twelfth polynucleotide is operably linked to an eighth RNA polymerase promoter and an eighth RNA polymerase terminator. The influenza virus genome segment includes the 3′ and 5′ packaging elements of vRNA of influenza virus hemagglutinin (HA) and includes a defect the coding sequence of influenza HA vRNA.

In some embodiments, a reporter gene may replace at least a portion of the coding sequence of an influenza vRNA. For example, in an exemplary embodiment, a reporter gene may replace at least a portion of the coding sequence of an influenza HA vRNA.

Any suitable reporter gene may be included. In some embodiments, the reporter gene may be a fluorescence gene including, for example, GFP, RFP, etc. In some embodiments, the reporter gene may include a luciferase enzyme, β-galactosidase, or chloramphenicol acetyltransferase.

In some embodiments, one or more of the promoters are inducible promoters. In some embodiments, one or more of the polynucleotides encoding an mRNA is operably linked to an inducible promoter. In some embodiments, each of the polynucleotides encoding a mRNA is operably linked to an inducible promoter. In some embodiments, the inducible promoters are preferably RNA Pol II promoters.

In some embodiments, one or more of the first inducible promoter, the second inducible promoter, and the third inducible promoter, and the fourth inducible promoter comprise different promoters. In some embodiments, each inducible promoter is a Pol II promoter.

In some embodiments, a polynucleotide that is operably linked to an inducible promoter (including, for example, an RNA Pol II promoter) is also operably linked to an RNA polymerase terminator. In some embodiments, the RNA polymerase terminator corresponds to the type (for example, Pol I, Pol II, or Pol III) of the inducible promoter. In some embodiments, the RNA polymerase terminator preferably includes a Pol II terminator.

Any suitable inducible promoter or combination of promoters may be used. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR.

The RNA polymerase promoters and RNA polymerase terminators for the polynucleotides encoding vRNA are further discussed below.

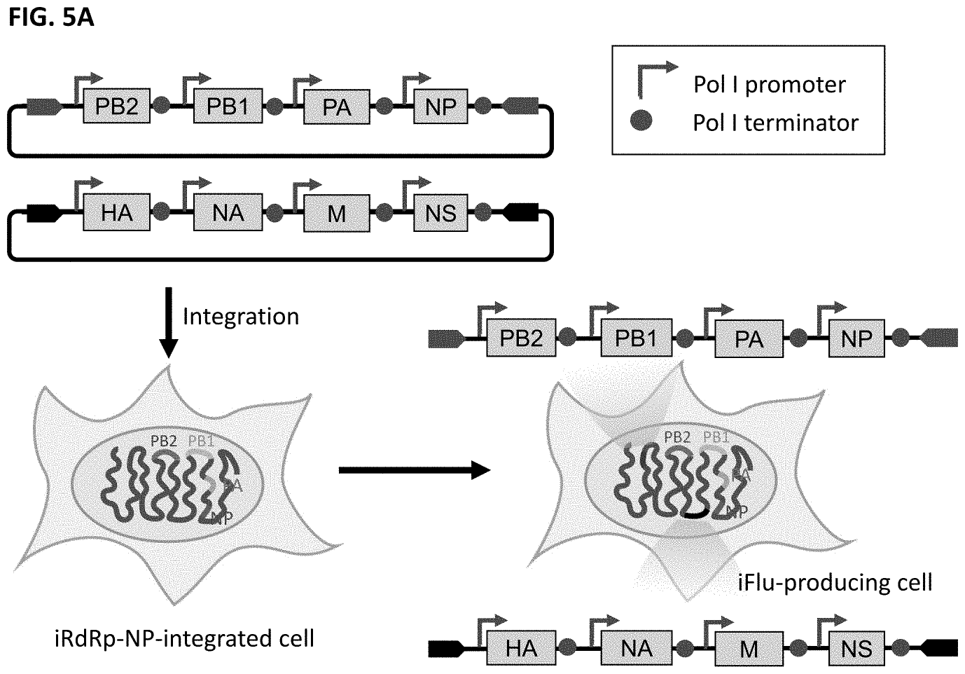

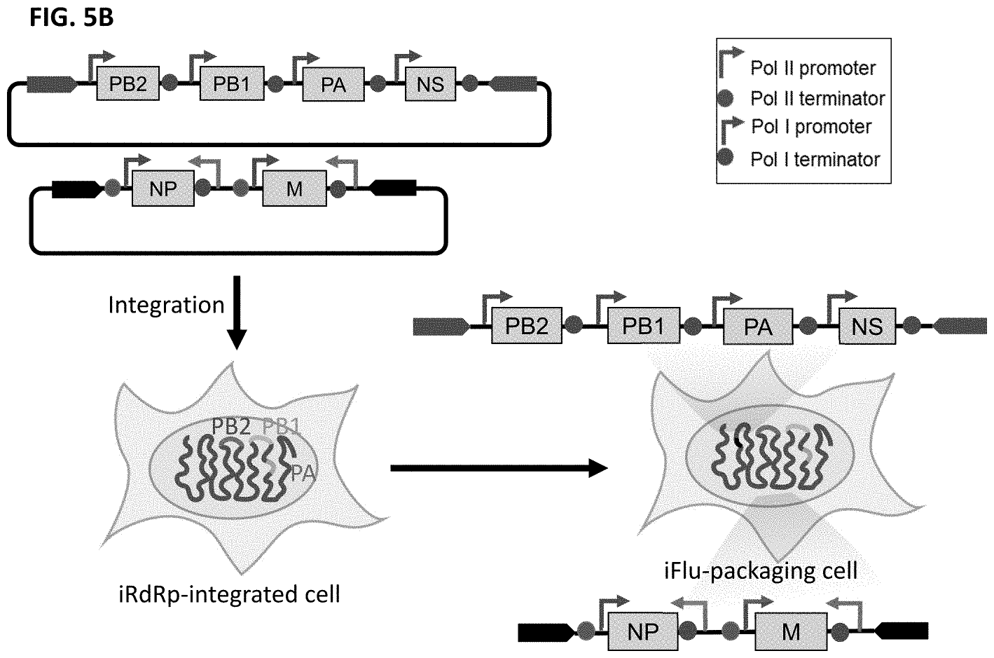

In other embodiments, a mammalian cell that produces a replication-deficient influenza virus may include a single polynucleotide for the mRNA and vRNA of PB1, PB2, PA, and NP, wherein each of those polynucleotides includes two separate promoters—one for the mRNA and one for the vRNA. (See, for example, FIG. 5A and FIG. 5B.) In some embodiments, the promoter for polynucleotide encoding the mRNA will be a Pol II promoter and the promoter for polynucleotide encoding the vRNA will be a Pol I promoter. The mammalian cell may further include additional polynucleotides for the vRNAs of the remaining influenza vRNAs. The coding sequence of at least one vRNAs is defective, however, resulting in a virus may be packaged only if a polynucleotide encoding the protein that would otherwise be encoded by the gene is supplied.

In an exemplary embodiment, a mammalian cell may include: a first polynucleotide encoding mRNA and vRNA of a PB1 subunit of RdRp, wherein the first polynucleotide is operably linked to a first promoter and a second promoter; a second polynucleotide encoding mRNA and vRNA of a PB2 subunit of RdRp, wherein the second polynucleotide is operably linked to a third promoter and a fourth promoter; a third polynucleotide encoding mRNA and vRNA of a PA subunit of RdRp, wherein the third polynucleotide is operably linked to a fifth promoter and a sixth promoter; a fourth polynucleotide encoding mRNA and vRNA of NP, wherein the fourth polynucleotide is operably linked to a seventh promoter and an eighth promoter; a fifth polynucleotide encoding vRNA of NS, wherein the fifth polynucleotide is operably linked to a ninth promoter; a sixth polynucleotide encoding vRNA of M, wherein the sixth polynucleotide is operably linked to a tenth promoter; a seventh polynucleotide encoding vRNA of NA, wherein the seventh polynucleotide is operably linked to a eleventh promoter; and an eighth polynucleotide encoding an influenza virus genome segment wherein the eighth polynucleotide is operably linked to a twelfth RNA polymerase promoter. The influenza virus genome segment includes the 3′ and 5′ packaging elements of vRNA of influenza virus hemagglutinin (HA) and includes a defect the coding sequence of influenza HA vRNA.

In some embodiments, a reporter gene may replace at least a portion of the coding sequence of an influenza vRNA. For example, in an exemplary embodiment, a reporter gene may replace at least a portion of the coding sequence of an influenza HA vRNA.

Any suitable reporter gene may be included. In some embodiments, the reporter gene may be a fluorescence gene including, for example, GFP, RFP, etc. In some embodiments, the reporter gene may include a luciferase enzyme, β-galactosidase, or chloramphenicol acetyltransferase.

In some embodiments, one or more of the promoters are inducible promoters. In some embodiments, one or more of the polynucleotides encoding an mRNA is operably linked to an inducible promoter. In some embodiments, each of the polynucleotides encoding a mRNA is operably linked to an inducible promoter. In some embodiments, the inducible promoters are preferably RNA Pol II promoters.

In some embodiments, one or more of the first promoter, the third promoter, the fifth promoter, and the seventh promoter include an inducible promoter. In some embodiments, each of the first promoter, the third promoter, the fifth promoter, and the seventh promoter includes an inducible promoter. In some embodiments, each of the first promoter, the third promoter, the fifth promoter, and the seventh promoter includes a different inducible promoter. In some embodiments, the inducible promoters (for example, the first promoter, the third promoter, the fifth promoter, or the seventh promoter) are preferably RNA Pol II promoters. In some embodiments, a polynucleotide that is operably linked to the first promoter, the third promoter, the fifth promoter, or the seventh promoter further is also operably linked to an RNA polymerase terminator. In some embodiments, the RNA polymerase terminator corresponds to the type (Pol I, Pol II, or Pol III) of the inducible promoter. In some embodiments, the RNA polymerase terminator preferably includes a Pol II terminator.

Any suitable inducible promoter or combination of promoters may be used. Exemplary inducible promoters and the corresponding inducers are shown in Table 2 and include TetOn, Cumate rcTA, Ecdysone, iDimerize Regulated Transcription System (P_{ZI}-1), GeneSwitch System/pSwitch, LacSwitchII, VAC, PEACE, and TraR.

In some embodiments, the second promoter, the fourth promoter, the sixth promoter, the eighth promoter, the ninth promoter, the tenth promoter, the eleventh promoter, and the twelfth promoter include an RNA polymerase promoter.

An RNA polymerase promoter may include any suitable RNA polymerase promoter. In some embodiments, the RNA polymerase promoter for a polynucleotide encoding a vRNA preferably includes a Pol I promoter. Pol I transcribes genes encoding ribosomal rRNA, facilitating the production of vRNA from a DNA sequence encoding the vRNA. In some embodiments, where the RNA polymerase promoter includes a Pol II promoter, the influenza virus genome segment may be operably linked to ribozymes as described in U.S. Pat. No. 7,723,094. In some embodiments, the RNA polymerase promoter includes a human or a mouse promoter. In an exemplary embodiment, the RNA polymerase promoter for a polynucleotide encoding a vRNA includes a human Pol I promoter.

In some embodiments a polynucleotide that is operably linked to the second promoter, the fourth promoter, the sixth promoter, the eighth promoter, the ninth promoter, the tenth promoter, the eleventh promoter, or the twelfth promoter is also operably linked to an RNA polymerase terminator.

An RNA polymerase terminator may include any suitable RNA polymerase terminator. In some embodiments, the RNA polymerase terminator corresponds to the type (Pol I, Pol II, or Pol III) of an RNA polymerase promoter. In some embodiments, the RNA polymerase terminator for a polynucleotide encoding a vRNA preferably includes a Pol I terminator. In some embodiments, the RNA polymerase terminator includes a human or a mouse terminator. In an exemplary embodiment, the RNA polymerase terminator for a polynucleotide encoding a vRNA includes a mouse Pol I terminator.

In some embodiments, the mammalian cell that produces a single-cycle influenza virus because it includes a defect the coding sequence of influenza HA vRNA further includes a polynucleotide encoding mRNA of HA. As described in an exemplary embodiment in Example 2, a defect the coding sequence of influenza HA vRNA means that HA protein must be separately provided to generate a viral particle—but the absence of the influenza HA vRNA means that any viral particle produced is single cycle, that is, it may infect cells but is not be able to package new viral particles unless HA is supplied.

### Methods of Making the System

In another aspect, this disclosure describes methods of making a mammalian cell or a system capable of generating influenza virus or influenza virus proteins, as described herein.

### Methods of Making the RdRp-Integrated Mammalian Cell and the RdRp-NP-Integrated Mammalian Cell

In another aspect, this disclosure describes methods of making an RdRp-integrated mammalian cell and methods of making an RdRp-NP-integrated mammalian cell. In some embodiments, the mammalian cell may be a cell of a mammalian cell line. Exemplary mammalian cell lines in include a mammalian cell line including a 293T cell, a Madin-Darby Canine Kidney (MDCK) cell, or a Vero cell, etc.

In some embodiments, polynucleotides encoding mRNA of the subunits of RdRp operably linked to inducible promoters or the subunits of both NP and RdRp operably linked to inducible promoters may be stably integrated into a mammalian cell using lentiviral integration.

In some embodiments, polynucleotides encoding mRNA of the subunits of RdRp operably linked to inducible promoters or the subunits of both NP and RdRp operably linked to inducible promoters may be stably integrated into a mammalian cell using a transposable element.

In some embodiments, polynucleotides including a packaging element of influenza virus may be incorporated into the mammalian cell. The polynucleotide including a packaging element of influenza virus may further include a reporter gene. For example, as shown in an exemplary embodiment in Example 1, a Minigenome including the packaging element of HA and GFP may be incorporated in the mammalian cell.

In a related embodiment, a method of making an RdRp-integrated mammalian cell or an RdRp-NP-integrated mammalian cell may further include transfecting the mammalian cell with a polynucleotide that includes a vRNA of influenza virus in which the coding sequence of the vRNA gene is replaced with a reporter gene and the packaging sequences of the gene are retained.

Additionally or alternatively, the method may include transfecting the mammalian cell with a polynucleotide that includes an mRNA of influenza virus NS, M, NA, NP, or HA.

### Methods of Making the Replication Competent and Single-Cycle Influenza Virus (IV)-Integrated Mammalian Cell Lines

In another aspect, this disclosure describes methods of making an Influenza Virus (IV)-integrated mammalian cell. In some embodiments, the mammalian cell may be a cell of a mammalian cell line. Exemplary mammalian cell lines in include a mammalian cell line including a 293T cell, a Madin-Darby Canine Kidney (MDCK) cell, or a Vero cell, etc.

In some embodiments, the mammalian cell line produces a replication competent influenza virus. In some embodiments, the mammalian cell line produces a replication-deficient influenza virus including, for example, a single-cycle influenza virus.

Polynucleotides encoding mRNA of the subunits of RdRp operably linked to an inducible promoters or the subunits of both NP and RdRp operably linked to an inducible promoters may be stably integrated into a mammalian cell line using any suitable means.

In some embodiments, polynucleotides encoding mRNA of the subunits of RdRp operably linked to an inducible promoters or the subunits of both NP and RdRp operably linked to an inducible promoters may be stably integrated into a mammalian cell line using lentiviral integration.

In some embodiments, polynucleotides encoding mRNA of the subunits of RdRp operably linked to an inducible promoters or the subunits of both NP and RdRp operably linked to an inducible promoters may be stably integrated into a mammalian cell line using a transposable element.

Polynucleotides encoding an mRNA of influenza virus NS, M, NA, NP, or HA, or polynucleotides encoding a vRNA, or polynucleotides encoding both an mRNA and a vRNA may be stably integrated into a mammalian cell line using any suitable means. Example 4A describes and FIG. 5A shows a schematic of the integration of 8 vRNA into cells that inducibly express RdRP and NP (iRdRp-NP-integrated cell) to create cells that produce flu virus. Example 4B describes and FIG. 5B shows a schematic of the integration of polynucleotides that encode vRNAs of PB2, PB1, PA, and NS and polynucleotides will encode for both vRNAs and mRNAs of NP and M into cells that inducibly express RdRP and NP (iRdRp-NP-integrated cell) to create cells that will produce flu virus when transfected with HA and NA.

In some embodiments, polynucleotides encoding an mRNA of influenza virus NS, M, NA, NP, or HA, or polynucleotides encoding a vRNA, or polynucleotides encoding both an mRNA and a vRNA may be transiently transfected. In some embodiments, polynucleotides encoding an mRNA of influenza virus NS, M, NA, NP, or HA, or polynucleotides encoding a vRNA, or polynucleotides encoding both an mRNA and a vRNA may be stably integrated into the genome of the mammalian cell line. Polynucleotides maybe stably integrated by any suitable method including, for example, by using lentiviral integration or using a transposable element or some combination thereof.

### Methods of Using the System

In another aspect, this disclosure describes methods of using a cell or a system as described herein to generate influenza virus or an influenza virus protein.

### Exemplary Methods of Using the RdRp-Integrated Mammalian Cell and RdRp-NP-Integrated Mammalian Cell

In another aspect, this disclosure describes methods of using the RdRp-integrated cell or RdRp-NP-integrated cell. In some embodiments, the mammalian cell may be a cell of a mammalian cell line. Exemplary mammalian cell lines in include a mammalian cell line including a 293T cell, a Madin-Darby Canine Kidney (MDCK) cell, or a Vero cell, etc.

In some embodiments, a method includes transfecting an RdRp-integrated cell with a polynucleotide including vRNA of PB1, PB2, PA, HA, NP, NA, M, or NS of influenza virus, or a combination thereof.

In some embodiments, a method includes transfecting an RdRp-integrated cell with polynucleotides including vRNAs of PB1, PB2, PA, NP, matrix protein (M), and non-structural protein (NS).

In some embodiments, a method includes transfecting an RdRp-integrated cell with polynucleotides including vRNAs of PB1, PB2, PA, NP, matrix protein (M), non-structural protein (NS), and neuraminidase (NA).

In some embodiments, a method includes transfecting an RdRp-integrated cell with polynucleotides including vRNAs of PB1, PB2, PA, NP, matrix protein (M), non-structural protein (NS), hemagglutinin (HA), and neuraminidase (NA).

In some embodiments, each polynucleotide including a vRNA is operably linked to an RNA polymerase promoter. In some embodiments, the RNA polymerase promoter for a polynucleotide including a vRNA preferably includes a Pol I promoter. In some embodiments, each polynucleotide including a vRNA is operably linked to an RNA polymerase terminator. In some embodiments, the RNA polymerase terminator for a polynucleotide including a vRNA preferably includes a Pol I terminator.

In some embodiments, the polynucleotides used to transfect an RdRp-integrated cell further include an mRNA of NP.

In some embodiments, the vRNA and the corresponding mRNA are included in the same polynucleotide. For example, in one embodiment, the vRNA and the mRNA of NP are included on the same polynucleotide and the polynucleotide is operably linked to a first RNA polymerase promoter at the 5′-end of the negative sense strand of the polynucleotide and to a second RNA polymerase promoter at the 5′-end of positive sense strand of the polynucleotide. In an exemplary embodiment, the RNA polymerase promoter at the 5′-end of the vRNA strand of the polynucleotide includes a Pol I promoter and the RNA polymerase promoter at the 5′-end of mRNA strand of the polynucleotide includes a Pol II promoter.

In an alternative embodiment, the vRNA and the mRNA of NP are included on two separate polynucleotides: for example, a first polynucleotide encoding vRNA of NP and second polynucleotide encoding mRNA of NP, wherein each polynucleotide is operably linked to an RNA polymerase promoter. In an exemplary embodiment, the polynucleotide encoding vRNA of NP is operably linked to a Pol I promoter and the second polynucleotide encoding mRNA of NP is operably linked to a Pol II promoter.

In some embodiments, the polynucleotides used to transfect an RdRp-integrated cell further include a polynucleotide encoding an influenza virus genome segment that includes the 3′ and 5′ packaging elements of an influenza virus vRNA.

In some embodiments, the influenza virus genome segment includes a reporter gene that replaces at least a portion of the coding sequence of the influenza virus vRNA while retaining the cis packaging elements for the influenza virus vRNA. Inclusion of the cis packaging elements for the vRNA may be used to promote packaging of the reporter gene. In some embodiments, the packaging elements include the 5′ and 3′ untranslated regions of the vRNA. In some embodiments, the packaging elements include the 5′ and 3′ untranslated regions of the vRNA and an additional bundling signal in the coding sequence of the vRNA.

Any suitable reporter gene may be included. In some embodiments, the reporter gene may be a fluorescence gene including, for example, GFP, RFP, etc. In some embodiments, the reporter gene may include a luciferase enzyme, β-galactosidase, or chloramphenicol acetyltransferase.

In some embodiments the influenza virus genome segment may be operably linked to an RNA polymerase promoter. In some embodiments, the RNA polymerase promoter includes a human or a mouse promoter. In an exemplary embodiment, the RNA polymerase promoter of the influenza virus genome segment includes a human Pol I promoter.

In some embodiments the influenza virus genome segment may be operably linked to an RNA polymerase terminator. In some embodiments, the RNA polymerase terminator includes a human or a mouse terminator. In an exemplary embodiment, the RNA polymerase terminator of the influenza virus genome segment includes a mouse Pol I terminator.

In some embodiments, the polynucleotides used to transfect an RdRp-integrated cell further include a polynucleotide including mRNA of influenza virus HA, wherein the polynucleotide is operably linked to a tenth RNA polymerase promoter. In some embodiments, the RNA polymerase terminator includes a Pol I or a Pol II promoter terminator. In an exemplary embodiment, the RNA polymerase promoter includes a Pol II promoter.

In some embodiments, the method includes exposing a mammalian cell of the mammalian cell line to inducers of the first inducible promoter, the second inducible promoter, and the third inducible promoter. IN some embodiments, the mammalian cell may be exposed to the inducers sequentially. The mammalian cell may be exposed to the inducers in any order. For example, the mammalian cell may be exposed to the inducer of the second inducible promoter, then the inducer of the first inducible promoter, then the inducer of the third inducible promoter.

Upon the induction of inducible promoters using the inducer or inducers, the controlled genes are active and viral particles are packaged. Without wishing to be bound by theory, it is believed that the dynamics of the induction and packaging of the virus may be controlled by adding the inducers sequentially.

In some embodiments, the method includes exposing a mammalian cell of the mammalian cell line to a cell expressing hemagglutinin (HA). For example, as described in Example 1, the cells may be overlaid with a cell expressing HA. In some embodiments, exposure to a cell expressing HA amplifies the production of viral particles.

In some embodiments, the method includes producing replication-deficient viral particles. In some embodiments, the method includes producing replication competent viral particles.

In some embodiments, the method includes isolating viral particles produced by a mammalian cell of the mammalian cell line. For example, isolating the viral particles may include detecting a protein encoded by the reporter gene or isolating the viral particles expressing a protein encoded by the reporter gene or both.

In some embodiments, the method may further include exposing a subject to the viral particles.

In some embodiments, the method may include using the mammalian cell line, a cell of the mammalian cell line, or the viral particle produced by the mammalian cell line for drug screening.

### Exemplary Methods of Using IV-Integrated Mammalian Cell Line

In another aspect, this disclosure describes methods of using an Influenza Virus-integrated mammalian cell.

In some embodiments, the method may include using the mammalian cell line or a cell of the mammalian cell line to produce a viral particle. In some embodiments, the viral particle may be replication competent. In some embodiments, the viral particle may be replication-deficient including, for example, a single-cycle influenza virus particle. In some embodiments, the viral particle may include a specific type of influenza virus (for example, Influenza A virus or Influenza B virus) or subtype of influenza virus.

The method may, in some embodiments, further include exposing a subject to the viral particles including, for example, in the context of a vaccine.

In some embodiments, the method may include using the mammalian cell line, a cell of the mammalian cell line, or the viral particle produced by the mammalian cell line for drug screening.

### EXEMPLARY EMBODIMENTS

### A. Exemplary Aspects of a RdRp-Integrated and RdRp-NP-Integrated Mammalian Cell Lines

A1. A mammalian cell line comprising:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter; and

a third polynucleotide encoding mRNA of a PA subunit of RdRp of influenza virus, wherein the third polynucleotide is operably linked to a third inducible promoter,

wherein the first polynucleotide, the second polynucleotide, and the third polynucleotide are stably integrated into the genome of the mammalian cell.

A2. The mammalian cell line of Aspect A1, wherein the first inducible promoter, the second inducible promoter, and the third inducible promoter comprise different promoters.
A3. The mammalian cell line of Aspect A1 or A2, wherein

the first inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR;

the second inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR; and

the third inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR.

A4. The mammalian cell line of any one of Aspects A1 to A3, the mammalian cell line further comprising:

one or more additional polynucleotides encoding mRNA of HA, NP, NA, M, or NS of influenza virus, or a combination thereof,

wherein the one or more additional polynucleotides are stably integrated into the genome of the mammalian cell.

A5. The mammalian cell line of any one of Aspects A1 to A4, the mammalian cell line further comprising:

a fourth polynucleotide encoding mRNA of influenza virus RNA-binding nucleoprotein (NP), wherein the fourth polynucleotide is stably integrated into the genome of the mammalian cell.

A6. The mammalian cell line of any one of Aspects A1 to A5, wherein the mammalian cell is transiently transfected with a polynucleotide encoding influenza virus mRNA.
A7. The mammalian cell line of any one of Aspects A1 to A6, wherein the mammalian cell is transiently transfected with a polynucleotide encoding mRNA of influenza virus RNA-binding nucleoprotein (NP).
A8. The mammalian cell line of any one of Aspects A4 to A7, wherein the one or more additional polynucleotides encoding mRNA comprise DNA.
A9. The mammalian cell line of any one of Aspects A1 to A8, the mammalian cell line further comprising:

one or more additional polynucleotides encoding vRNA of PB1, PB2, PA, HA, NP, NA, M, or NS of influenza virus, or a combination thereof,

wherein the one or more additional polynucleotides are stably integrated into the genome of the mammalian cell.

A10. The mammalian cell line of any one of Aspects A1 to A9, wherein the mammalian cell is transiently transfected with one or more additional polynucleotides encoding vRNA of PB1, PB2, PA, HA, NP, NA, M, or NS of influenza virus, or a combination thereof.
A11. The mammalian cell line of Aspect A9 or A10, wherein the one or more additional polynucleotides encoding vRNA comprise DNA.
A12. The mammalian cell line of any one of Aspects A9 to A11, wherein one or more additional polynucleotides encoding vRNA is operably linked to an RNA polymerase promoter.
A13. The mammalian cell line of Aspect A12, wherein the RNA polymerase promoter comprises a Pol I promoter.
A14. The mammalian cell line of any one of Aspects A9 to A13, wherein one or more additional polynucleotides encoding vRNA is operably linked to an RNA polymerase terminator.
A15. The mammalian cell line of any one of Aspects A1 to A14, wherein the mammalian cell is transiently transfected with a polynucleotide comprising an influenza virus genome segment,

wherein the influenza virus genome segment comprises 3′ and 5′ packaging elements of an influenza virus vRNA;

wherein the influenza virus genome segment comprises a reporter gene that replaces at least a portion of a coding sequence of the influenza virus vRNA; and

wherein the influenza virus genome segment is operably linked to an RNA polymerase promoter.

A16. The mammalian cell line of Aspect A15, wherein the RNA polymerase promoter comprises a Pol I promoter.
A17. The mammalian cell line of Aspect A15 or A16, wherein the reporter gene comprises a fluorescence gene including, a luciferase enzyme, β-galactosidase, or chloramphenicol acetyltransferase.
A18. The mammalian cell line of any one of Aspects A15 to A17, wherein the reporter gene comprises GFP.
A19. The mammalian cell line of any one of Aspects A15 to A18, wherein the influenza virus genome segment is operably linked to an RNA polymerase terminator.
A20. The mammalian cell line of any one of Aspects A15 to A19, wherein the influenza virus vRNA encodes at least a portion of HA.
A21. The mammalian cell line of any one of Aspects A1 to A20, wherein influenza virus comprises Influenza A virus.
A22. The mammalian cell line of any one of Aspects A1 to A21, wherein the first polynucleotide, the second polynucleotide, and the third polynucleotide comprise DNA and encode positive-sense RNA.

### B. Exemplary Embodiments: Methods of Using the Mammalian Cell Lines

B1. A method of using the mammalian cell line of any one of the Exemplary Embodiments of a RdRp-Integrated and RdRp-NP-Integrated Mammalian Cell Lines (Aspects A1 to A22).
B2. The method of Aspect B1, wherein the method comprises transiently transfecting the mammalian cell line of any one of Aspects A1 to A22 with one or more polynucleotides encoding vRNA of PB1, PB2, PA, HA, NP, NA, M, or NS of influenza virus, or a combination thereof.
B3. The method of Aspect B1 or B2, wherein the method comprises stably integrating into the mammalian cell line of any one of Aspects A1 to A22 with one or more polynucleotides encoding vRNA of PB1, PB2, PA, HA, NP, NA, M, or NS of influenza virus, or a combination thereof.
B4. The method of any one of Aspects B1 to B3, wherein the method comprises transfecting the mammalian cell line of any one of Aspects A1 to A22 with polynucleotides comprising:

a first polynucleotide encoding vRNA of a PB1 subunit of RNA-dependent RNA polymerase (RdRp) of influenza virus, wherein the first polynucleotide is operably linked to a first RNA polymerase promoter;

a second polynucleotide encoding vRNA of PB2 subunit of RdRp of influenza virus, wherein the second polynucleotide is operably linked to a second RNA polymerase promoter;

a third polynucleotide encoding vRNA of PA subunit of RdRp of influenza virus, wherein the third polynucleotide is operably linked to a third RNA polymerase promoter;

a fourth polynucleotide encoding vRNA of influenza virus matrix protein (M), wherein the fourth polynucleotide is operably linked to a fourth RNA polymerase promoter;

a fifth polynucleotide encoding vRNA of influenza virus non-structural protein (NS), wherein the fifth polynucleotide is operably linked to a fifth RNA polymerase promoter; and

a sixth polynucleotide encoding vRNA of influenza virus neuraminidase (NA), wherein the sixth polynucleotide is operably linked to a sixth RNA polymerase promoter.

B5. The method of any one of Aspects B1 to B4, wherein the influenza virus comprises Influenza A Virus.
B6. The method of Aspect B4 or B5, wherein the first RNA polymerase promoter, the second RNA polymerase promoter, the third RNA polymerase promoter, the fourth RNA polymerase promoter, the fifth RNA polymerase promoter, and the sixth RNA polymerase promoter comprise a Polymerase I (Pol I) promoter.
B7. The method of any one of Aspects B4 to B6, wherein the polynucleotides further comprise:

a seventh polynucleotide encoding vRNA of influenza virus NP and mRNA of influenza virus NP,

wherein the seventh polynucleotide is operably linked to a seventh RNA polymerase promoter at the 5′-end of the negative sense strand of the polynucleotide and an eighth RNA polymerase promoter at the 5′-end of positive sense strand of the polynucleotide.

B8. The method of any one of Aspects B4 to B7, wherein the polynucleotides further comprise:

a seventh polynucleotide encoding vRNA of influenza virus NP, and

an eighth polynucleotide encoding mRNA of influenza virus NP,

wherein the seventh polynucleotide is operably linked to a seventh RNA polymerase promoter and the eighth polynucleotide is operably linked to an eighth RNA polymerase promoter.

B9. The method of Aspect B7 or B8, wherein the seventh RNA polymerase promoter comprises a Polymerase I promoter and the eighth RNA polymerase promoter comprises a Polymerase II (pol II) promoter.
B10. The method of any one of Aspects B4 to B9, wherein the polynucleotides further comprise a ninth polynucleotide encoding an influenza virus genome segment,

wherein the influenza virus genome segment comprises 3′ and 5′ packaging elements of an influenza virus vRNA,

wherein the influenza virus genome segment comprises a reporter gene that replaces at least a portion of a coding sequence of the influenza virus vRNA, and

wherein the transcription of the influenza virus genome segment is operably linked to a ninth RNA polymerase promoter.

B11. The method of Aspect B10, wherein the ninth RNA polymerase promoter comprises a Pol I promoter.
B12. The method of Aspect B10 or B11, wherein the reporter gene comprises GFP.
B13. The method of any one of Aspects B10 to B12, wherein the influenza virus vRNA comprises HA.
B14. The method of any one of Aspects B4 to B13, wherein the polynucleotides further comprise:

a tenth polynucleotide encoding mRNA of influenza virus HA,

wherein the tenth polynucleotide is operably linked to a tenth RNA polymerase promoter.

B15. The method of Aspect B14, wherein the tenth RNA polymerase promoter comprises a Polymerase II (Pol II) promoter.
B16. The method of any one of Aspects B4 to B16, wherein one or more of the first polynucleotide, second polynucleotide, third polynucleotide, fourth polynucleotide, fifth polynucleotide, sixth polynucleotide, seventh polynucleotide, eighth polynucleotide, ninth polynucleotide, or tenth polynucleotide is operably linked to a polymerase terminator.
B17. The method of any one of Aspects B4 to B16, the method comprising sequentially exposing a mammalian cell of the mammalian cell line to inducers of the first inducible promoter, the second inducible promoter, and the third inducible promoter.
B18. The method of any one of Aspects B1 to B17, the method comprising:

generating an infectious influenza virus particle, or

generating an influenza virus protein.

B19. The method of any one of Aspects B1 to B18, the method comprising exposing a mammalian cell of the mammalian cell line to a cell expressing hemagglutinin (HA), and wherein the method further comprises producing an influenza virus particle.
B20. The method of Aspect B18 or B19, wherein the influenza virus particle is a replication-deficient virus particle.
B21. The method of any one of Aspects B18 to B20, wherein the method further comprises isolating an influenza virus particle produced by a mammalian cell of the mammalian cell line.
B22. The method of Aspect B21, wherein the method comprises isolating the influenza virus particle, wherein the influenza virus particle comprises a protein encoded by a reporter gene.
B23. The method of any one of Aspects B18 to B22 wherein the method further comprises exposing a subject to the influenza virus particle.
B24. The method of any one of Aspects B18 to B22 wherein the method further comprises using the influenza virus particle for drug screening.

### C-1. Exemplary Embodiments: Methods of Making an iRdRp-Integrated Mammalian Cell Line

C1-1. A method comprising stably integrating into a mammalian cell:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter.

C1-2. The method of Aspect C1-1, wherein the method comprises lentiviral integration of one or more of the first polynucleotide, the second polynucleotide, and the third polynucleotide.
C1-3. The method of Aspect C1-1 or C1-2, wherein the method comprises integration of one or more of the first polynucleotide, the second polynucleotide, and the third polynucleotide using a transposable element.
C1-4. The method of any one of Aspects C1-1 to C1-3 wherein the method comprises transfecting the mammalian cell with a polynucleotide, the polynucleotide comprising a packaging element of influenza virus.
C1-5. The method of Aspect C1-4, wherein the polynucleotide comprising a packaging element of influenza virus further comprises a reporter gene.
C1-6. The method of any one Aspects C1-1 to C1-5 wherein the method comprises transfecting the mammalian cell with a polynucleotide, the polynucleotide comprising a vRNA of influenza virus.
C1-7. The method of any one Aspects C1-1 to C1-6 wherein the method comprises transfecting the mammalian cell with a polynucleotide, the polynucleotide comprising an mRNA of influenza virus NS, M, NA, NP, or HA.
C1-8. The method of any one of Aspects C1-1 to C1-7, the method comprising stably integrating into the mammalian cell one or more additional polynucleotides, the one or more additional polynucleotide encoding vRNA of PB1, PB2, PA, HA, NP, NA, M, or NS of influenza virus, or a combination thereof.
C1-9. The method of any one Aspects C1-1 to C1-8, wherein the influenza virus comprises Influenza A virus.

### C-2. Exemplary Embodiments: Methods of Making an iRdRp-NP-Integrated Mammalian Cell

C2-1. A method comprising stably integrating into a mammalian cell:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter; and

a fourth polynucleotide encoding mRNA of influenza virus RNA-binding nucleoprotein (NP).

C2-2. The method of Aspect C2-1, wherein the method comprises lentiviral integration of one or more of the first polynucleotide, the second polynucleotide, the third polynucleotide, and the fourth polynucleotide.
C2-3. The method of Aspect C2-1 or C2-2, wherein the method comprises integration of one or more of the first polynucleotide, the second polynucleotide, the third polynucleotide, and the fourth polynucleotide using a transposable element.
C2-4. The method of any one of Aspects C2-1 to C2-3 wherein the method comprises transfecting the mammalian cell with a polynucleotide, the polynucleotide comprising a packaging element of influenza virus.
C2-5. The method of Aspect C2-4, wherein the polynucleotide comprising a packaging element of influenza virus further comprises a reporter gene.
C2-6. The method of any one of Aspects C2-1 to C2-5 wherein the method comprises transfecting the mammalian cell with a polynucleotide, the polynucleotide comprising a vRNA of influenza virus.
C2-7. The method of any one of Aspects C2-1 to C2-6 wherein the method comprises transfecting the mammalian cell with a polynucleotide, the polynucleotide comprising an mRNA of influenza virus NS, M, NA, NP, or HA.
C2-8. The method of any one of Aspects C2-1 to C2-7, the method comprising stably integrating into the mammalian cell one or more additional polynucleotides, the one or more additional polynucleotide encoding vRNA of PB1, PB2, PA, HA, NP, NA, M, or NS of influenza virus, or a combination thereof.
C2-9. The method of any one Aspects C2-1 to C2-8, wherein the influenza virus comprises Influenza A virus.

### D-1. Exemplary Embodiments of a Replication Competent Influenza Virus (IV)-Integrated Mammalian Cell Line

D1-1. A mammalian cell line comprising:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter;

a fourth polynucleotide encoding mRNA of influenza virus RNA-binding nucleoprotein (NP), wherein the fourth polynucleotide is operably linked to a fourth inducible promoter;

a fifth polynucleotide encoding vRNA of influenza virus NP, wherein the fifth polynucleotide is operably linked to a first RNA polymerase promoter and a first RNA polymerase terminator;

a sixth polynucleotide encoding vRNA of influenza virus non-structural protein (NS), wherein the sixth polynucleotide is operably linked to a second RNA polymerase promoter and a second RNA polymerase terminator;

a seventh polynucleotide encoding vRNA of influenza virus matrix protein (M), wherein the seventh polynucleotide is operably linked to a third RNA polymerase promoter and a third RNA polymerase terminator;

an eighth polynucleotide encoding vRNA of a PB1 subunit of RdRp of influenza virus, wherein the eighth polynucleotide is operably linked to a fourth RNA polymerase promoter and a fourth RNA polymerase terminator;

a ninth polynucleotide encoding vRNA of a PB2 subunit of RdRp of influenza virus, wherein the ninth polynucleotide is operably linked to a fifth RNA polymerase promoter and a fifth RNA polymerase terminator;

a tenth polynucleotide encoding vRNA of a PA subunit of RdRp of influenza virus, wherein the tenth polynucleotide is operably linked to a sixth RNA polymerase promoter and a sixth RNA polymerase terminator;

D1-2. The mammalian cell line of Aspect D1-1 further comprising:

an eleventh polynucleotide encoding vRNA of influenza virus neuraminidase (NA), wherein the eleventh polynucleotide is operably linked to a seventh RNA polymerase promoter and a seventh RNA polymerase terminator; or

a twelfth polynucleotide encoding vRNA of influenza virus hemagglutinin (HA), wherein the twelfth polynucleotide is operably linked to an eighth RNA polymerase promoter and an eighth polymerase terminator; or

both.

D1-3. The mammalian cell line of Aspect D1-1 or D1-2, wherein the first polynucleotide, the second polynucleotide, the third polynucleotide, or the fourth polynucleotide, or any combination thereof are stably integrated into the genome of the mammalian cell.
D1-4. The mammalian cell line of any one of Aspects D1-1 to D1-3, wherein the first inducible promoter, the second inducible promoter, and the third inducible promoter, and the fourth inducible promoter comprise different promoters.
D1-5. The mammalian cell line of any one of Aspects D1-1 to D1-4, wherein

the first inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR;

the second inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR;

the third inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR; or

the fourth inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR.

D1-6. The mammalian cell line of any one of Aspects D1-1 to D1-5, wherein the mammalian cell line is transiently transfected with the fifth polynucleotide, the sixth polynucleotide, the seventh polynucleotide, the eighth polynucleotide, the ninth polynucleotide, the tenth polynucleotide, the eleventh polynucleotide, and the twelfth polynucleotide.
D1-7. The mammalian cell line of any one of Aspects D1-1 to D1-5, wherein the fifth polynucleotide, the sixth polynucleotide, the seventh polynucleotide, the eighth polynucleotide, the ninth polynucleotide, the tenth polynucleotide, the eleventh polynucleotide, or the twelfth polynucleotide, or any combination thereof are stably integrated into the genome of the mammalian cell line.
D1-8. The mammalian cell line of any one of Aspects D1-1 to D1-5, wherein the fifth polynucleotide, the sixth polynucleotide, the seventh polynucleotide, the eighth polynucleotide, the ninth polynucleotide, the tenth polynucleotide, the eleventh polynucleotide, and the twelfth polynucleotide are stably integrated into the genome of the mammalian cell line.
D1-9. The mammalian cell line of any one of Aspects D1-1 to D1-8, wherein the RNA polymerase promoter comprises a human Pol I promoter.
D1-10. The mammalian cell line of any one of Aspects D1-1 to D1-9, wherein the RNA polymerase terminator comprises a murine Pol I terminator.
D1-11. A mammalian cell line comprising:

a first polynucleotide encoding mRNA and vRNA of a PB1 subunit of RNA-dependent RNA polymerase (RdRp) of influenza virus, wherein the first polynucleotide is operably linked to a first promoter and a second promoter;

a second polynucleotide encoding mRNA and vRNA of a PB2 subunit of RdRp of influenza virus, wherein the second polynucleotide is operably linked to a third promoter and a fourth promoter;

a third polynucleotide encoding mRNA and vRNA of a PA subunit of RdRp of influenza virus, wherein the third polynucleotide is operably linked to a fifth promoter and a sixth promoter;

a fourth polynucleotide encoding mRNA and vRNA of influenza virus RNA-binding nucleoprotein (NP), wherein the fourth polynucleotide is operably linked to a seventh promoter and an eighth promoter;

a fifth polynucleotide encoding vRNA of influenza virus non-structural protein (NS), wherein the fifth polynucleotide is operably linked to a ninth promoter;

a sixth polynucleotide encoding vRNA of influenza virus hemagglutinin (HA), wherein the sixth polynucleotide is operably linked to a tenth promoter;

a seventh polynucleotide encoding vRNA of influenza virus matrix protein (M), wherein the seventh polynucleotide is operably linked to a eleventh promoter;

an eighth polynucleotide encoding vRNA of influenza virus neuraminidase (NA), wherein the eighth polynucleotide is operably linked to a twelfth promoter.

D1-12. The mammalian cell line of Aspect D1-11, wherein the first polynucleotide, the second polynucleotide, and the third polynucleotide, the fourth polynucleotide, the fifth polynucleotide, the sixth polynucleotide, the seventh polynucleotide, or the eighth polynucleotide, or any combination thereof are stably integrated into the genome of the mammalian cell.
D1-13. The mammalian cell line of Aspect D1-11 or D1-12, wherein one or more of the first promoter, the third promoter, the fifth promoter, or the seventh promoter comprise inducible promoters.
D1-14. The mammalian cell line of any one of Aspects D1-11 to D1-13, wherein the first promoter, the third promoter, the fifth promoter, and the seventh promoter comprise different promoters.
D1-15. The mammalian cell line of Aspect D1-13 or D1-14, wherein the inducible promoters are selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR.
D1-16. The mammalian cell line of any one of Aspects D1-11 to D1-15, wherein the second promoter, the fourth promoter, the sixth promoter, the eighth promoter, the ninth promoter, the tenth promoter, the eleventh promoter, and the twelfth promoter comprise a Pol I promoter.
D1-17. The mammalian cell line of any one of Aspects D1-1 to D1-16 wherein

the first polynucleotide further comprises the eighth polynucleotide;

the second polynucleotide further comprises the ninth polynucleotide;

the third polynucleotide further comprises the tenth polynucleotide; and

a fourth polynucleotide further comprises the fifth polynucleotide.

D1-18. The mammalian cell line of any one of Aspects D1-1 to D1-17 wherein the influenza virus comprises Influenza A virus.

### D-2. Exemplary Embodiments of a Single-Cycle Influenza Virus (IV)-Integrated Mammalian Cell Line

D2-1. A mammalian cell line comprising:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter;

a fourth polynucleotide encoding mRNA of influenza virus RNA-binding nucleoprotein (NP), wherein the fourth polynucleotide is operably linked to a fourth inducible promoter;

a fifth polynucleotide encoding vRNA of influenza virus NP, wherein the fifth polynucleotide is operably linked to a first RNA polymerase promoter and a first RNA polymerase terminator;

a sixth polynucleotide encoding vRNA of influenza virus non-structural protein (NS), wherein the sixth polynucleotide is operably linked to a second RNA polymerase promoter and a second RNA polymerase terminator;
a seventh polynucleotide encoding vRNA of influenza virus matrix protein (M), wherein the seventh polynucleotide is operably linked to a third RNA polymerase promoter and a third RNA polymerase terminator;

an eighth polynucleotide encoding vRNA of influenza virus neuraminidase (NA), wherein the eighth polynucleotide is operably linked to a fourth RNA polymerase promoter and a fourth RNA polymerase terminator;

a ninth polynucleotide encoding vRNA of a PB1 subunit of RdRp of influenza, wherein the ninth polynucleotide is operably linked to a fifth RNA polymerase promoter and a fifth RNA polymerase terminator;

a tenth polynucleotide encoding vRNA of a PB2 subunit of RdRp of influenza, wherein the tenth polynucleotide is operably linked to a sixth RNA polymerase promoter and a sixth RNA polymerase terminator;

an eleventh polynucleotide encoding vRNA of a PA subunit of RdRp of influenza, wherein the eleventh polynucleotide is operably linked to a seventh RNA polymerase promoter and a seventh RNA polymerase terminator;

a twelfth polynucleotide encoding an influenza virus genome segment wherein the twelfth polynucleotide is operably linked to an eighth RNA polymerase promoter and an eighth RNA polymerase terminator,
wherein the influenza virus genome segment comprises 3′ and 5′ packaging elements of vRNA of influenza virus hemagglutinin (HA),
wherein the influenza virus genome segment comprises a defect in the coding sequence of influenza HA vRNA.
   D2-2. The mammalian cell line of Aspect D2-1, wherein the first polynucleotide, the second polynucleotide, and the third polynucleotide are stably integrated into the genome of the mammalian cell line.
   D2-3. The mammalian cell line of Aspect D2-1, wherein the first polynucleotide, the second polynucleotide, the third polynucleotide, the fourth polynucleotide are stably integrated into the genome of the mammalian cell line.
   D2-4. The mammalian cell line of any one of Aspects D2-1 to D2-3, wherein the first inducible promoter, the second inducible promoter, the third inducible promoter, and the fourth inducible promoter comprise different promoters.
   D2-5. The mammalian cell line of any one of Aspects D2-1 to D2-4, wherein a reporter gene replaces at least a portion of the coding sequence of influenza HA vRNA.
   D2-6. The mammalian cell line of Aspect D2-5, wherein the reporter gene comprises GFP.
   D2-7. The mammalian cell line of any one of Aspects D2-1 to D2-6, wherein

the first inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR;

the second inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR

the third inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR; or

the fourth inducible promoter is selected from a promoter comprising TetOn, Cumate rcTA, Ecdysone, iDimerize Regulated Transcription System (P_{ZI}-1), GeneSwitch System/pSwitch, LacSwitchII, VAC, PEACE, or TraR.

D2-8. The mammalian cell line of any one of Aspects D2-1 to D2-7, wherein the mammalian cell line is transiently transfected with the fifth polynucleotide, the sixth polynucleotide, the seventh polynucleotide, the eighth polynucleotide, the ninth polynucleotide, the tenth polynucleotide, the eleventh polynucleotide, and the twelfth polynucleotide.
D2-9. The mammalian cell line of any one of Aspects D2-1 to D2-7, wherein the fifth polynucleotide, the sixth polynucleotide, the seventh polynucleotide, the eighth polynucleotide, the ninth polynucleotide, the tenth polynucleotide, the eleventh polynucleotide, or the twelfth polynucleotide are stably integrated into the genome of the mammalian cell line.
D2-10. The mammalian cell line of any one of Aspects D2-1 to D2-7, wherein the fifth polynucleotide, the sixth polynucleotide, the seventh polynucleotide, the eighth polynucleotide, the ninth polynucleotide, the tenth polynucleotide, the eleventh polynucleotide, and the twelfth polynucleotide are stably integrated into the genome of the mammalian cell line.
D2-11. The mammalian cell line of any one of Aspects D2-1 to D2-10, wherein the RNA polymerase promoter comprises a human Pol I promoter.
D2-12. The mammalian cell line of any one of Aspects D2-1 to D2-11, wherein the RNA polymerase terminator comprises a murine Pol I terminator.
D2-13. A mammalian cell line comprising:

a first polynucleotide encoding mRNA and vRNA of a PB1 subunit of RNA-dependent RNA polymerase (RdRp) of influenza virus, wherein the first polynucleotide is operably linked to a first promoter and a second promoter;

a second polynucleotide encoding mRNA and vRNA of a PB2 subunit of RdRp of influenza virus, wherein the second polynucleotide is operably linked to a third promoter and a fourth promoter;

a third polynucleotide encoding mRNA and vRNA of a PA subunit of RdRp of influenza virus, wherein the third polynucleotide is operably linked to a fifth promoter and a sixth promoter;

a fourth polynucleotide encoding mRNA and vRNA of influenza virus RNA-binding nucleoprotein (NP), wherein the fourth polynucleotide is operably linked to a seventh promoter and an eighth promoter;

a fifth polynucleotide encoding vRNA of influenza virus non-structural protein (NS), wherein the fifth polynucleotide is operably linked to a ninth promoter;

a sixth polynucleotide encoding vRNA of influenza virus matrix protein (M), wherein the sixth polynucleotide is operably linked to a tenth promoter;

a seventh polynucleotide encoding vRNA of an influenza virus neuraminidase (NA), wherein the seventh polynucleotide is operably linked to a eleventh promoter; and

an eighth polynucleotide encoding an influenza virus genome segment wherein the eighth polynucleotide is operably linked to a twelfth RNA polymerase promoter,
wherein the influenza virus genome segment comprises 3′ and 5′ packaging elements of vRNA of influenza virus hemagglutinin (HA),
wherein the influenza virus genome segment comprises a defect the coding sequence of influenza HA vRNA.
   D2-14. The mammalian cell line of Aspect D2-13, wherein the first polynucleotide, the second polynucleotide, and the third polynucleotide, the fourth polynucleotide, the fifth polynucleotide, the sixth polynucleotide, the seventh polynucleotide, and the eighth polynucleotide are stably integrated into the genome of the mammalian cell line.
   D2-15. The mammalian cell line of Aspect D2-13 or D2-14, wherein one or more of the first promoter, the third promoter, the fifth promoter, or the seventh promoter comprise inducible promoters.
   D2-16. The mammalian cell line of any one of Aspects D2-13 to D2-15, wherein the first inducible promoter, the third inducible promoter, the fifth inducible promoter, and the seventh promoter comprise different promoters.
   D2-17. The mammalian cell line of any one of Aspects D2-13 to D2-16, wherein the seventh promoter comprises a Pol II promoter.
   D2-18. The mammalian cell line of any one of Aspects D2-13 to D2-17, wherein the second promoter, the fourth promoter, the sixth promoter, the eighth promoter, the ninth promoter, the tenth promoter, the eleventh promoter, and the twelfth promoter comprise a Pol I promoter.
   D2-19. The mammalian cell line of any one of Aspects D2-1 to D2-18, wherein a reporter gene replaces at least a portion of the coding sequence of influenza HA vRNA.
   D2-20. The mammalian cell line of Aspect D2-19, wherein the reporter gene comprises a fluorescence gene including, a luciferase enzyme, β-galactosidase, or chloramphenicol acetyltransferase.
   D2-21. The mammalian cell line of Aspect D2-20, wherein the reporter gene comprises GFP.
   D2-22. The mammalian cell line of any one of Aspects D2-1 to D2-21, the mammalian cell line further comprising a polynucleotide encoding mRNA of influenza virus hemagglutinin (HA).
   D2-23. The mammalian cell line of any one of Aspects D2-1 to D2-12, wherein the influenza virus comprises Influenza A virus.

### E-1. Exemplary Embodiments: Methods of Using Replication Competent IV-Integrated Mammalian Cell Lines

E1-1. A method of using the mammalian cell line of any one of the Aspects D1-1 to D1-18.
E1-2. The method of Aspect E1-1, the method comprising:

generating an infectious influenza virus particle, wherein the influenza virus particle is optionally a replication-deficient influenza virus particle, or

generating an influenza virus protein.

E1-3. The method of Aspect E1-1 or E1-2, the method comprising exposing a mammalian cell of the mammalian cell line to inducers of the first inducible promoter, the second inducible promoter, the third inducible promoter, and the fourth inducible promoter.
E1-4. The method of Aspect E1-3, wherein the method comprises sequentially exposing the mammalian cell of the mammalian cell line to inducers.
E1-5. The method of any one of Aspects E1-1 to E1-4, the method comprising exposing a mammalian cell of the mammalian cell line to a cell expressing hemagglutinin (HA), and wherein the method further comprises producing viral particles.
E1-6. The method of any one of Aspects E1-1 to E1-5, wherein the method comprises isolating viral particles produced by the mammalian cell line.
E1-7. The method of Aspect E1-5 or E1-6, wherein the viral particles comprise replication competent viral particles.
E1-8. The method of any one of Aspects E1-5 to E1-7, wherein the method further comprises exposing a subject to the viral particles.
E1-9. The method of any one of Aspects E1-5 to E1-7, wherein the method further comprises using the viral particles for drug screening.

### E-2. Exemplary Embodiments: Methods of Using Single-Cycle Influenza Virus (IV)-Integrated Mammalian Cell Line

E2-1. A method of using the mammalian cell of any one of the Aspects D2-1 to D2-23.
E2-2. The method of Aspect E1-1, the method comprising:

generating an infectious influenza virus particle, wherein the influenza virus particle is optionally a replication-deficient influenza virus particle, or

generating an influenza virus protein.

E2-3. The method of Aspect E1-1 or E1-2, the method comprising exposing a mammalian cell of the mammalian cell line to inducers of the first inducible promoter, the second inducible promoter, the third inducible promoter, and the fourth inducible promoter.
E2-4. The method of Aspect E1-3, wherein the method comprises sequentially exposing the mammalian cell of the mammalian cell line to inducers.
E2-5. The method of any one of Aspects E2-1 to E2-4, the method comprising exposing a mammalian cell of the mammalian cell line to a cell expressing hemagglutinin (HA), and wherein the method further comprises producing viral particles.
E2-6. The method of any one of Aspects E2-1 to E2-5, wherein the method comprises isolating viral particles produced by the mammalian cell line.
E2-7. The method of Aspect E2-5 or E2-6, wherein the viral particles comprise single-cycle viral particles.
E2-8. The method of any one of Aspects E2-5 to E2-7, wherein the method further comprises exposing a subject to the viral particles.
E2-9. The method of any one of Aspects E2-5 to E2-7, wherein the method further comprises using the viral particles for drug screening.

### F-1. Exemplary Embodiments: Methods of Making a Replication Competent Influenza Virus (IV)-Integrated Mammalian Cell

F1-1. A method comprising stably integrating into a mammalian cell:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter;

a fourth polynucleotide encoding mRNA of influenza virus RNA-binding nucleoprotein (NP), wherein the fourth polynucleotide is operably linked to a fourth inducible promoter;

a fifth polynucleotide encoding vRNA of influenza virus NP, wherein the fifth polynucleotide is operably linked to a first RNA polymerase promoter and a first RNA polymerase terminator;
a sixth polynucleotide encoding vRNA of influenza virus non-structural protein (NS), wherein the sixth polynucleotide is operably linked to a second RNA polymerase promoter and a second RNA polymerase terminator;

a seventh polynucleotide encoding vRNA of influenza virus matrix protein (M), wherein the seventh polynucleotide is operably linked to a third RNA polymerase promoter and a third RNA polymerase terminator;

an eighth polynucleotide encoding vRNA of a PB1 subunit of RdRp of influenza, wherein the eighth polynucleotide is operably linked to a fourth RNA polymerase promoter and a fourth RNA polymerase terminator;

a ninth polynucleotide encoding vRNA of a PB2 subunit of RdRp of influenza, wherein the ninth polynucleotide is operably linked to a fifth RNA polymerase promoter and a fifth RNA polymerase terminator; and

a tenth polynucleotide encoding vRNA of a PA subunit of RdRp of influenza, wherein the tenth polynucleotide is operably linked to a sixth RNA polymerase promoter and a sixth RNA polymerase terminator.
F1-2. The method of Aspect F1-1, the method further comprising stably integrating into the mammalian cell:

an eleventh polynucleotide encoding vRNA of influenza virus neuraminidase (NA), wherein the eleventh polynucleotide is operably linked to a seventh RNA polymerase promoter and a seventh RNA polymerase terminator; or

a twelfth polynucleotide encoding vRNA of influenza virus hemagglutinin (HA), wherein the twelfth polynucleotide is operably linked to an eighth RNA polymerase promoter and an eighth polymerase terminator; or

both.

F1-3. The method of Aspect F1-1 or F1-2, wherein the method comprises integration of one or more of first polynucleotide, the second polynucleotide, the third polynucleotide, the fourth polynucleotide, the fifth polynucleotide, the sixth polynucleotide, the seventh polynucleotide, the eighth polynucleotide, the ninth polynucleotide, the tenth polynucleotide, the eleventh polynucleotide, or the twelfth polynucleotide using a lentivirus and/or a transposable element.
F1-4. A method comprising stably integrating into a mammalian cell

a first polynucleotide encoding mRNA and vRNA of a PB1 subunit of RNA-dependent RNA polymerase (RdRp) of influenza virus, wherein the first polynucleotide is operably linked to a first promoter and a second promoter;

a second polynucleotide encoding mRNA and vRNA of a PB2 subunit of RdRp of influenza virus, wherein the second polynucleotide is operably linked to a third promoter and a fourth promoter;

a third polynucleotide encoding mRNA and vRNA of a PA subunit of RdRp of influenza virus, wherein the third polynucleotide is operably linked to a fifth promoter and a sixth promoter;

a fourth polynucleotide encoding mRNA and vRNA of influenza virus RNA-binding nucleoprotein (NP), wherein the fourth polynucleotide is operably linked to a seventh promoter and an eighth promoter;

a fifth polynucleotide encoding vRNA of influenza virus non-structural protein (NS), wherein the fifth polynucleotide is operably linked to a ninth promoter;

a sixth polynucleotide encoding vRNA of influenza virus hemagglutinin (HA), wherein the sixth polynucleotide is operably linked to a tenth promoter;

a seventh polynucleotide encoding vRNA of influenza virus matrix protein (M), wherein the seventh polynucleotide is operably linked to an eleventh promoter;

an eighth polynucleotide encoding vRNA of influenza virus neuraminidase (NA), wherein the eighth polynucleotide is operably linked to a twelfth promoter.

F1-5. The method of Aspect F1-4, wherein the method comprises lentiviral integration of one or more of the first polynucleotide, the second polynucleotide, the third polynucleotide, the fourth polynucleotide, the fifth polynucleotide, the sixth polynucleotide, the seventh polynucleotide, or the eighth polynucleotide.
F1-6. The method of Aspect F1-4 or F1-5, wherein the method comprises integration of one or more of first polynucleotide, the second polynucleotide, the third polynucleotide, the fourth polynucleotide, the fifth polynucleotide, the sixth polynucleotide, the seventh polynucleotide, or the eighth polynucleotide using a transposable element.
F1-7. The method of any one of Aspects F1-1 to F1-6 wherein the influenza virus comprises Influenza A virus.

### F-2. Exemplary Embodiments: Methods of Making a Single-Cycle Influenza Virus (IV)-Integrated Cell

F2-1. A method comprising stably integrating into a mammalian cell:

a first polynucleotide encoding mRNA of a PB1 subunit of RNA-dependent RNA polymerase (RdRp) of influenza virus, wherein the first polynucleotide is operably linked to a first inducible promoter;

a second polynucleotide encoding mRNA of a PB2 subunit of RdRp of influenza virus, wherein the second polynucleotide is operably linked to a second inducible promoter;

a third polynucleotide encoding mRNA of a PA subunit of RdRp of influenza virus, wherein the third polynucleotide is operably linked to a third inducible promoter;

a fourth polynucleotide encoding mRNA of influenza virus RNA-binding nucleoprotein (NP), wherein the fourth polynucleotide is operably linked to a fourth inducible promoter;

a fifth polynucleotide encoding vRNA of influenza virus NP, wherein the fifth polynucleotide is operably linked to a first RNA polymerase promoter and a first RNA polymerase terminator;

a sixth polynucleotide encoding vRNA of influenza virus non-structural protein (NS), wherein the sixth polynucleotide is operably linked to a second RNA polymerase promoter and a second RNA polymerase terminator;

a seventh polynucleotide encoding vRNA of influenza virus matrix protein (M), wherein the seventh polynucleotide is operably linked to a third RNA polymerase promoter and a third RNA polymerase terminator;

an eighth polynucleotide encoding vRNA of influenza virus neuraminidase (NA), wherein the eighth polynucleotide is operably linked to a fourth RNA polymerase promoter and a fourth RNA polymerase terminator;

a ninth polynucleotide encoding vRNA of a PB1 subunit of RdRp of influenza, wherein the ninth polynucleotide is operably linked to a fifth RNA polymerase promoter and a fifth RNA polymerase terminator;

a tenth polynucleotide encoding vRNA of a PB2 subunit of RdRp of influenza, wherein the tenth polynucleotide is operably linked to a sixth RNA polymerase promoter and a sixth RNA polymerase terminator;

an eleventh polynucleotide encoding vRNA of a PA subunit of RdRp of influenza, wherein the eleventh polynucleotide is operably linked to a seventh RNA polymerase promoter and a seventh RNA polymerase terminator;

a twelfth polynucleotide encoding an influenza virus genome segment wherein the twelfth polynucleotide is operably linked to an eighth RNA polymerase promoter and an eighth RNA polymerase terminator,
wherein the influenza virus genome segment comprises 3′ and 5′ untranslated regions (UTRs) of vRNA of influenza virus hemagglutinin (HA),
wherein the influenza virus genome segment comprises a reporter gene that replaces at least a portion of a coding sequence of influenza HA vRNA.
   F2-2. The method of Aspect F2-1, wherein the method comprises lentiviral integration of one or more of the first polynucleotide, the second polynucleotide, the third polynucleotide, the fourth polynucleotide, the fifth polynucleotide, the sixth polynucleotide, the seventh polynucleotide, the eighth polynucleotide, the ninth polynucleotide, the tenth polynucleotide, the eleventh polynucleotide, or the twelfth polynucleotide.
   F2-3. The method of Aspect F2-1 or F2-2, wherein the method comprises integration of one or more of first polynucleotide, the second polynucleotide, the third polynucleotide, the fourth polynucleotide, the fifth polynucleotide, the sixth polynucleotide, the seventh polynucleotide, the eighth polynucleotide, the ninth polynucleotide, the tenth polynucleotide, the eleventh polynucleotide, or the twelfth polynucleotide using a transposable element.
   F2-4. A method comprising stably integrating into a mammalian cell:

a first polynucleotide encoding mRNA and vRNA of a PB1 subunit of RNA-dependent RNA polymerase (RdRp) of influenza virus, wherein the first polynucleotide is operably linked to a first promoter and a second promoter;

a second polynucleotide encoding mRNA and vRNA of a PB2 subunit of RdRp of influenza virus, wherein the second polynucleotide is operably linked to a third promoter and a fourth promoter;

a third polynucleotide encoding mRNA and vRNA of a PA subunit of RdRp of influenza virus, wherein the third polynucleotide is operably linked to a fifth promoter and a sixth promoter;

a fourth polynucleotide encoding mRNA and vRNA of influenza virus RNA-binding nucleoprotein (NP), wherein the fourth polynucleotide is operably linked to a seventh promoter and an eighth promoter;

a fifth polynucleotide encoding vRNA of influenza virus non-structural protein (NS), wherein the fifth polynucleotide is operably linked to a ninth promoter;

a sixth polynucleotide encoding vRNA of influenza virus matrix protein (M), wherein the sixth polynucleotide is operably linked to a tenth promoter;

a seventh polynucleotide encoding vRNA of influenza virus neuraminidase (NA), wherein the seventh polynucleotide is operably linked to an eleventh promoter;

an eighth polynucleotide encoding an influenza virus genome segment wherein the eighth polynucleotide is operably linked to a twelfth RNA polymerase promoter,
wherein the influenza virus genome segment comprises 3′ and 5′ untranslated regions (UTRs) of vRNA of influenza virus hemagglutinin (HA),
wherein the influenza virus genome segment comprises a reporter gene that replaces at least a portion of a coding sequence of influenza HA vRNA.
   F2-5. The method of Aspect F2-4, wherein the method comprises lentiviral integration of one or more of the first polynucleotide, the second polynucleotide, the third polynucleotide, the fourth polynucleotide, the fifth polynucleotide, the sixth polynucleotide, the seventh polynucleotide, or the eighth polynucleotide.
   F2-6. The method of Aspect F2-4 or F2-5, wherein the method comprises integration of one or more of first polynucleotide, the second polynucleotide, the third polynucleotide, the fourth polynucleotide, the fifth polynucleotide, the sixth polynucleotide, the seventh polynucleotide, or the eighth polynucleotide using a transposable element.
   F2-7. The method of any one of Aspects F2-1 to F2-6, the method further comprising stably integrating into the mammalian cell a polynucleotide encoding mRNA of influenza virus hemagglutinin (HA).
   F2-8. The method of any one of Aspects F2-1 to F2-6, the method further comprising transiently transfecting the mammalian cell with a polynucleotide encoding mRNA of influenza virus hemagglutinin (HA).
   F2-9. The mammalian cell line of any one of Aspects F2-1 to F2-8, wherein the influenza virus comprises Influenza A virus.

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, Mo.) and were used without further purification unless otherwise indicated.

### Example 1—Construction of Integrated Inducible RdRp Cells (iRdRp Cells)

This Example describes construction of inducible RdRp (iRdRP) cells, wherein RdRP is integrated into a mammalian cell genome.

### Packaging of Lentivirus

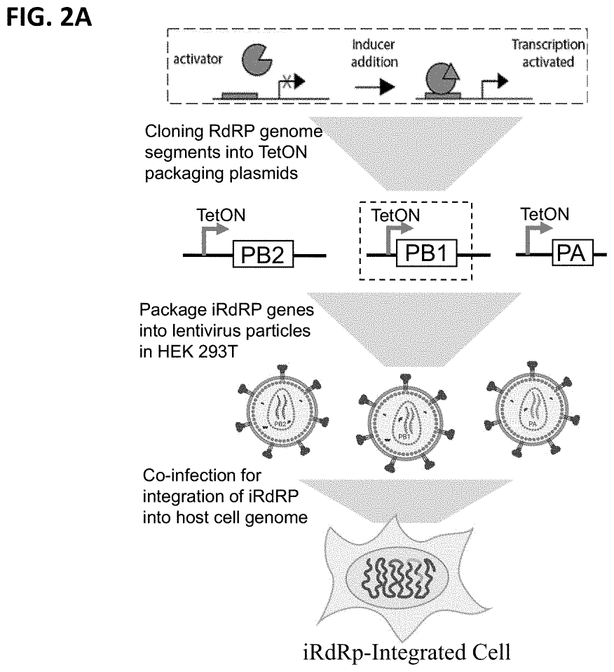

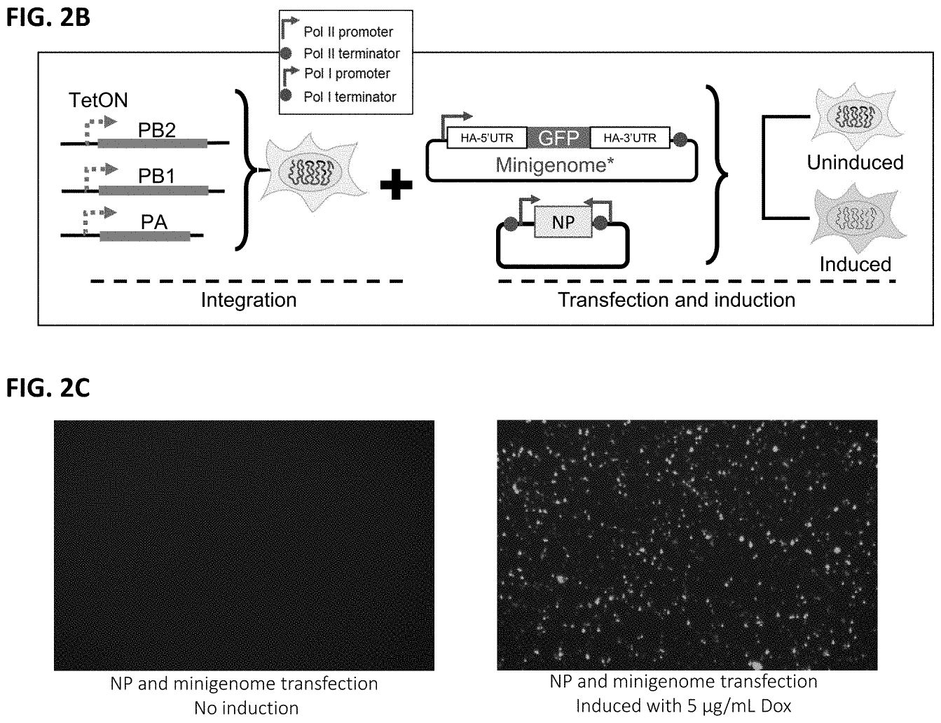

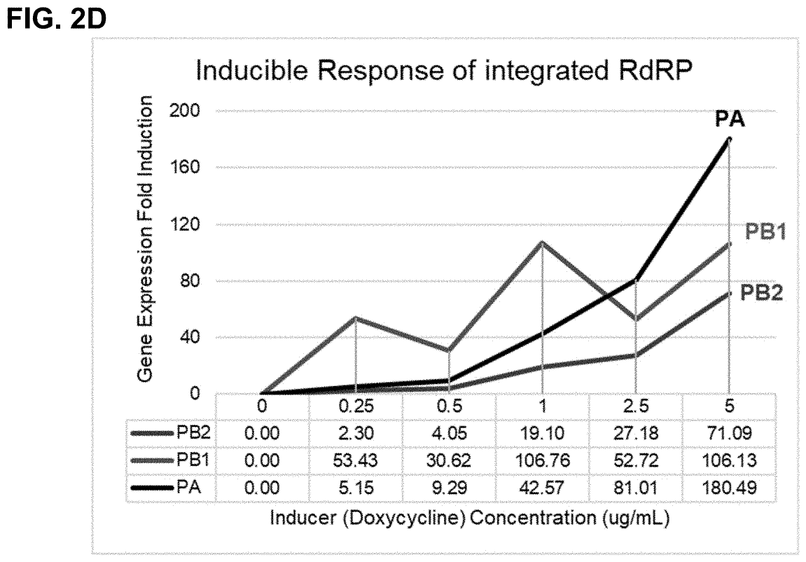

Plasmids contain puromycin resistance gene (puromycin N-acetyltransferase) or blasticidin S resistance gene (bsr) and each of the RdRP genes (PB2, PB1, PA) were each packaged an into lentiviral vectors including: pLenti-TetON-PB2, pLenti-TetON-PB1, pLenti-TetON-PA (see FIG. 2A). In this example, all viral genes are from IAV strain A/Puerto Rico/8/1934. The sequences of pLenti-TetON-PB2, pLenti-TetON-PB1, pLenti-TetON-PA are shown in Table 1. The packaging of Lentiviruses was performed according to the protocols described by Invitrogen using Lipofectamine 3000 Transfection Reagent (L3000015). Briefly, 293T cells were co-transfected with a mixture of plasmids including (1) the packaging vector psPAX (Addgene ID 12260) (2) the envelope vector pMD2.G (Addgene ID 12259), and (3) the transfer plasmid, which is one of the pLenti-TetON-PB2, pLenti-TetON-PB1, or pLenti-TetON-PA. The viral supernatant was collected at 24 hours and 52 hours post transfection, then filtered through 0.45-um filter (Millipore-Sigma). The lentiviruses were then transduced into 293T host cells. Cells with genome-integrated genes were selected using puromycin or bsr resistance.

Although for the purposes of this Example, the same inducible promoter (TetOn) was used for each of PB1, PB2, and PA, any suitable inducible promoter could be used. Additional exemplary promoters and the corresponding inducer of each promoter are shown in Table 2.

TABLE 2
Exemplary inducible promoters and corresponding inducers.

| Inducible promoter system | Inducer |
|---|---|
| TetON | Tetracycline, Doxycycline |
| Cumate rcTA | Cumate |
| Ecdysone | Ponasterone A |
| iDimerize Regulated Transcription | A/C Heterodimerizer |
| System (P_{ZI}-1) | |
| GeneSwitch System/pSwitch | Mifepristone |
| LacSwitchII | β-D-thiogalactopyranoside (ITPG) |
| VAC | Vanillic Acid |
| PEACE | Phloretin |
| TraR | 3-Oxo-C8-HSL |

### Construction of RdRp Expressing Cell Lines

As shown in FIG. 2A, RdRp-expressing cell lines were constructed by the co-infection of three lentiviral vectors (pLenti-TetON-PB2, pLenti-TetON-PB1, and pLenti-TetON-PA) that also contain a puromycin resistance gene into HEK 293T host cells following the protocol described in Elegheert, et al. (Elegheert et al. Nat Protoc 13, 2991-3017 (2018)).

Infected cells were then subjected to selection for integrated cells by being cultured in medium containing 10 ug/mL puromycin. All puromycin-resistant cells were then single-cell cloned by limiting dilution in 96-well plate. All single-cell clones were scanned for the presence of either one, two, or three genes inside the genome using a minigenome assay with proper complementary genes, as described in to Velthuis et al., Assays to Measure the Activity of Influenza Virus Polymerase, in Influenza Virus: Methods and Protocols, Y. Yamauchi, Editor 2018, Springer New York: New York, N.Y. p. 343-374. Briefly, the minigenome assay was conducted by transfection of a minigenome and NP. The minigenome included the packaging elements of an HA gene in which the coding sequence of the gene was replaced by GFP. (See (Breen et al. Viruses 8, 179 (2016)), especially FIG. 1.) The NP gene was under the control of a Pol II promoter.

Out of 94 single-cell clones screened, 5 clones had PB1 and PA integrated and 3 clones had PB2 and PB1 integrated. Clones with PB1 and PA integrated were then infected with lentiviral vector containing TetON-PB2, while PB2- and PB1-positive clones were re-infected with TetON-PA. The resulting clones mentioned above were blasticidin S selected and screened again using the minigenome assay, as described above to identify seven clones that have all three RdRp genes (PB1, PB2, and PA) integrated. These clones were cultured for further studies. A cell expressing all three RdRp genes is hereinafter referred to as an “iRdRp” cell.

Functional Assay to Demonstrate Inducible Activity of iRdRP Cells

To demonstrate inducible activity of iRdRP cells, the minigenome assay was conducted as described in to Velthuis et al., Assays to Measure the Activity of Influenza Virus Polymerase, in Influenza Virus: Methods and Protocols, Y. Yamauchi, Editor 2018, Springer New York: New York, N.Y. p. 343-374. As shown in FIG. 2B, iRdRP cells were transfected with a plasmid containing NP and the minigenome plasmid described above using Lipofectamine 3000 (Invitrogen, L3000001). Transfected cells were induced by Doxycycline at concentration of 5 ug/mL or remained uninduced at the time of transfection as a negative control. Transfected cells were imaged and harvested for qRT-PCR after 36-48 hours post transfection. Results of imaging are shown in FIG. 2C.

qRT-PCR were performed as previously described (Nolan et al. Nat Protoc 1, 1559-1582 (2006)). RNAs from different samples were extracted using RNeasy Mini Kit (Cat. #74504, Qiagen, Hilden, Germany) and used as template to synthesize cDNA using First-Strand Synthesis Supermix (Cat. #11752050, Invitrogen, Carlsbad, Calif.). cDNAs are used as templates for qPCR using SYBR Green PCR Master Mix (Cat. #4364344, Applied Biosystems, Foster City, Calif.). Transcript expression of PB2, PB1, PA were normalized to the expression of the housekeeping GAPDH. The expression of mRNA of PA, PB1 and PB2 upon induction were confirmed by the results of qRT-PCR. Results of qPCR are shown in FIG. 2D.

### Example 2—Production of Infectious Single-Cycle HA-GFP Flu Virus from the iRdRP Cells

This Example describes using iRdRP cells to produce a single-cycle infectious influenza virus particle.

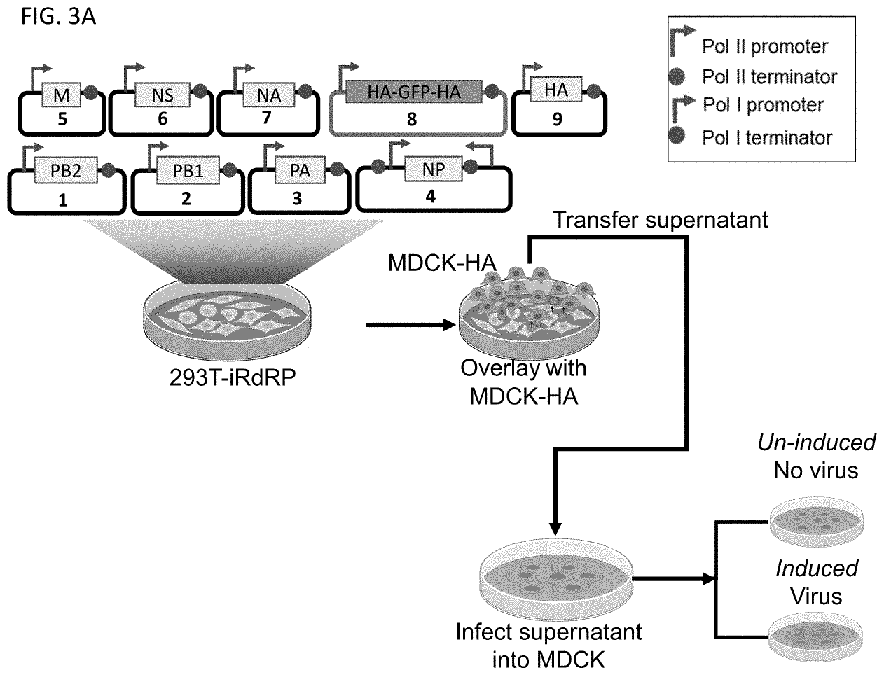

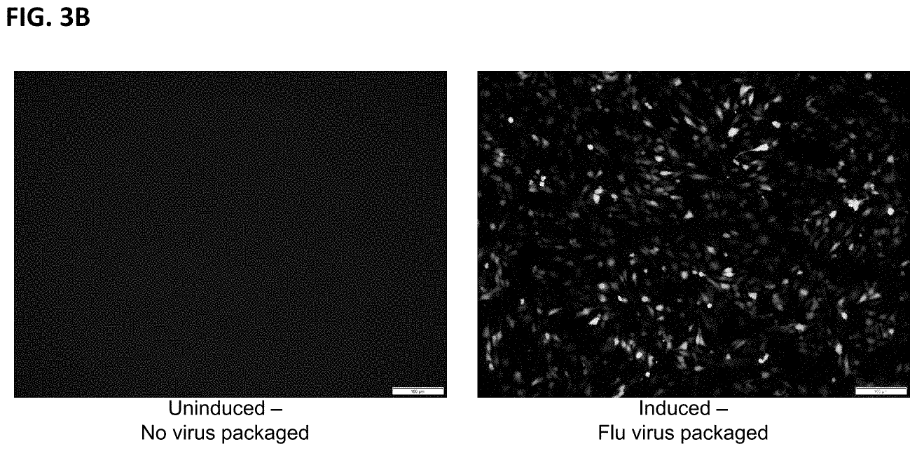

iRdRP was co-transfected with of a combination of plasmids: six plasmids (labeled 1-3 and 5-7 in FIG. 3A) each encoding for vRNA of one of PA, PB1, PB2, NA, M, NS; a minigenome including a modified vRNA of HA in which the HA protein coding sequence was replaced with GFP (HA-GFP-HA) (labeled 8 in FIG. 3A); pCAGGS-HA (labeled 9 in FIG. 3A) and pDZ-NP (labeled 4 in FIG. 3A). In this example, all viral genes were from IAV strain A/Puerto Rico/8/1934.

Each of six vRNA genes (PA, PB1, PB2, NA, M, and NS) were separately cloned in a pPol I vector in which the gene transcription is driven by a human Pol I promoter and murine Pol I terminator. (See FIG. 3A, plasmid 1-3 and 5-7.) A minigenome including a modified HA construct—in which the coding sequence of the HA gene was replaced by GFP—was also cloned into a pPol I vector in which the gene transcription is driven by a human Pol I promoter and murine Pol I terminator. (See FIG. 3A, plasmid 8.)

Because the coding region of the HA gene was replaced by GFP, HA protein was provided by transfection with pCAGGS-HA which includes a polynucleotide sequence encoding mRNA of HA. Note that the iRdRP was not transfected with a construct encoding a function vRNA for HA.

The pDZ-NP vector (see FIG. 3A, plasmid 4) encodes for both vRNA and mRNA of NP. vRNA of NP is used for viral genome packaging, and mRNA of NP allows for NP protein expression. In combination with the mRNA of PA, PB1 and PB2 expressed upon induction of the iRdRP cell, the expression of mRNA for NP results in a functional RdRP.

The viral particles released were expected to have seven normal viral genome segments (PA, PB1, PB2, NA, M, NS, and NP) and a defective HA genome segment.

By including pCAGGS-HA (plasmid 8)—which includes a polynucleotide sequence encoding mRNA of HA—in the iRdRP cell, infectious virus may be produced. However, because the cell does not produce any HA vRNA, the virus produced may infect other cells and replicate the viral genome, but it can only be packaged in cells expressing HA (Fodor et al. J Virol 73, 9679-9682 (1999), Gao et al. J Virol 82, 6419-6426 (2008)).

To detect the infectious virus produced in iRdDP cells, 24 hours after transfection, the transfected cells were overlaid by MDCK-HA cells, as described in Sjaastad et al. (Sjaastad et al. Proc Natl Acad Sci USA 115, 9610-9615 (2018)) to amplify the population of flu virus particles packaged in iRdRP cells for the sake of detection. The supernatant of the transfected and overlaid cells was harvested 48-52 hours after overlaying with MDCK-HA. The supernatant was then used to infect a culture of MDCK cells (which did not express HA). Any MDCK cell infected by the HA-GFP virus in the supernatant turned green. The results, shown in FIG. 3B, indicate that with induction, the iRdRP cells produced HA-GFP flu virus particles, as shown by the number of MDCK cells turned green after infection with supernatant (FIG. 3B, right panel). Without induction there no flu virus was packaged (FIG. 3B, left panel).

### Example 3—Production of Infectious Replication-Competent Flu Virus from the iRdRP Cells

This Example describes using iRdRP cells to produce replication-competent infectious influenza virus particle virus.

The production of fully replication-competent flu virus in iRdRP cells was conducted as previously described (Fodor et al. J Virol 73, 9679-9682 (1999), Martinez-Sobrido et al. J Vis Exp 42, 2057 (2010)). iRdRP cells were transfected with seven pPolI plasmids encoding for seven vRNAs (PA, PB1, PB2, NA, M, NS, and HA) and the pDZ-NP plasmid (as described in Example 2). In this example, pDZ-NP plasmid encodes for NP gene either from IAV strain A/Puerto Rico/8/1934 (denoted as NP-PR8), or strain A/WSN/1933 (denoted as NP-WSN). All the other plasmids in this Example encode for viral genes from IAV strain A/Puerto Rico/8/1934. 24 hours post-transfection, the transfected cells were overlaid with MDCK to amplify the number of virus particles produced in the transfection. 48-52 hours post-overlaying, the supernatant was harvested.

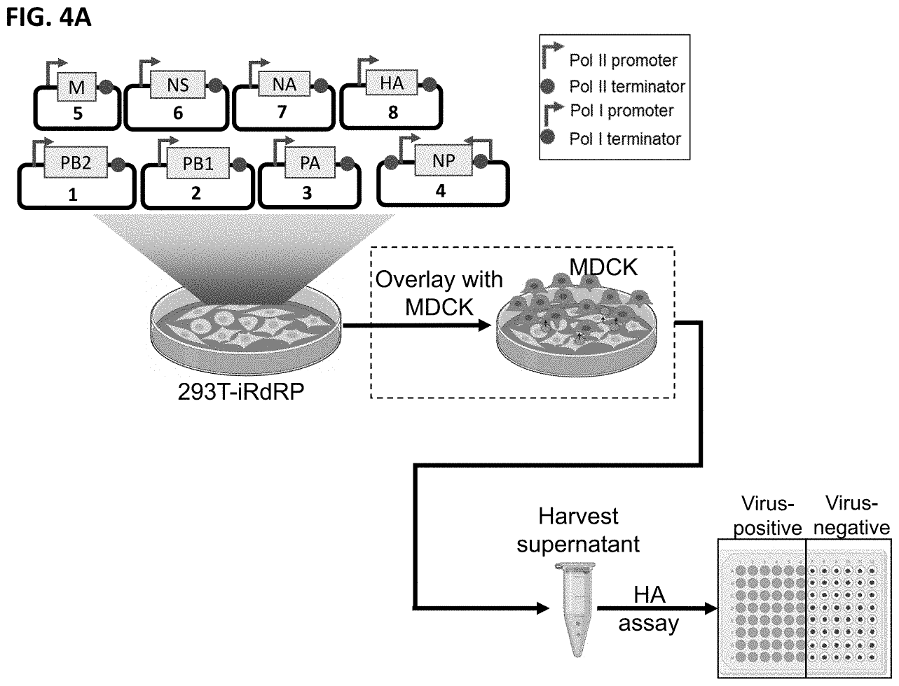

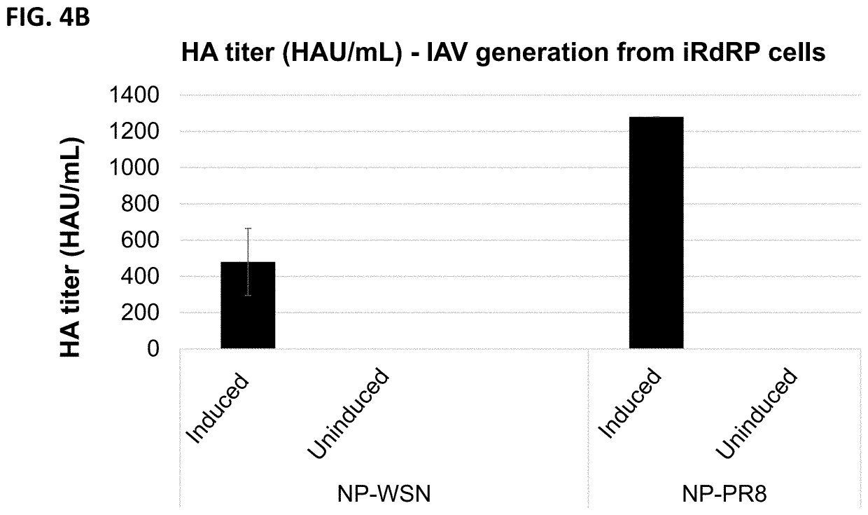

An HA assay was performed on the supernatant to detect the presence of flu virus. The HA assay was performed following the protocol of Killian, Hemagglutination Assay for Influenza Virus, in Animal Influenza Virus, E. Spackman, Editor 2014, Springer New York: New York, N.Y. p. 3-9. Briefly, HA proteins on the surface of virus particles bind to sialic acid receptors of red blood cells. This binding causes the agglutination of the red blood cells and prevents them from settling out of suspension. Negative samples, without HA, showed settlement of red blood cells at the bottom of the well; while in the case of positive samples, the uniform reddish suspension of red blood cells indicates the presence of infectious virus (FIG. 4A). The samples may be serially-diluted to determine the virus titer, as known as HA titer (HAU/mL).

These results demonstrate that virus particles may be packaged inducibly—because, as shown in FIG. 4B, virus particles were detected only in samples packaged in induced iRdRP cells. When NP gene from another virus strain was used, iRdRP cells was still package virus particles inducibly, although at a lower efficiency, indicating the versatility for generating virus.

### Example 4A

This Prophetic Example describes construction of an inducible influenza virus (iIV) mammalian cell line, wherein each vRNA will be integrated into a mammalian cell genome.

An iRdRp cell, made as described in Example 1, will be stably transfected with a polynucleotide encoding mRNA of influenza virus RNA-binding nucleoprotein (NP). The polynucleotide encoding mRNA of NP will be stably integrated into the genome of the mammalian cell line to provide an iRdRp-NP-integrated cell.

As shown in FIG. 5A, the iRdRp-NP-integrated cell will be stably transfected with polynucleotides encoding vRNA of PB1, PB2, PA, NP, HA, NA, M, and NS. The polynucleotides encoding the vRNA will be operably linked to a RNA polymerase promoter and terminator (for example, Pol I). The polynucleotides encoding the vRNAs will be stably integrated into the genome of the mammalian cell line to provide an iFlu-producing cell.

The iFlu-producing cells will be able to produce flu virus only under induced conditions; the induction profiles may be modified and optimized.

TABLE 1
Sequences of inducible RdRP (iRdRP) constructs

| | Bold = TetON promoter | |
|---|---|---|
| TetON-PB2 | 5′gagtttactccctatcagtgatagagaacgtatgtcgagtttactccctatcagtgatagagaacgatg | SEQ ID NO: 1 |
| (double | tcgagtttactccctatcagtgatagagaacgtatgtcgagtttactccctatcagtgatagagaacgtat | |
| underlined = | gtcgagtttactccctatcagtgatagagaacgtatgtcgagtttatccctatcagtgatagagaacgtat | |
| PB2) | gtcgagutactccctatcagtgatagagaacgtatgtcgaggtaggcgtgtacggtgggaggcctatataa | |
| | gcagagctcgtttagtgaaccgtcagatcgcctggagaattggctagcGCGATCGCCTAGGAATTCGTCGA | |
| | CCTGCAGGTTTAAACggATCagcgaaagcaggtcaattatattcaatatggaaagaataaaagaactaaga | |
| | aatctaatgtcgcagtctcgcacccgcgagatactcacaaaaaccaccgtggaccatatggccataatcaa | |
| | gaagtacacatcaggaagacaggagaagaacccagcacttaggatgaaatggatgatggcaatgaaatatc | |
| | caattacagcagacaagaggataacggaaatgattcctgagagaaatgagcaaggacaaactttatggagt | |
| | aaaatgaatgatgcaggatcagaccgagtgatggtatcacctctggctgtgacatggtggaataggaatgg | |
| | accaataacaaatacagttcattatccaaaaatctacaaaacttattttgaaagagtcgaaaggctaaagc | |
| | atggaacctttggccctgtccattttagaaaccaagtcaaaatacgtcggagagttgacataaatcctggt | |
| | catgcagatctcagtgccaaggaggcacaggatgtaatcatggaagttgttttccctaacgaagtgggagc | |
| | caggatactaacatcggaatcgcaactaacgataaccaaagagaagaaagaagaactccaggattgcaaaa | |
| | tttctcctttgatggttgcatacatgttggagagagaactggtccgcaaaacgagattcctcccagtggct | |
| | ggtggaacaagcagtgtgtacattgaagtgttgcatttgactcaaggaacatgctgggaacagatgtatac | |
| | tccaggaggggaagtgaggaatgatgatgttgatcaaagcttgattattgctgctaggaacatagtgagaa | |
| | gagctgcagtatcagcagatccactagcatctttattggagatgtgccacagcacacagattggtggaatt | |
| | aggatggtagacatccttaggcagaacccaacagaagagcaagccgtggatatatgcaaggctgcaatggg | |
| | actgagaattagctcatccttcagttttggtggattcacatttaagagaacaagcggatcatcagtcaaga | |
| | gagaggaagaggtgcttacgggcaatcttcaaacattgaagataagagtgcatgagggatatgaagagttc | |
| | acaatggttgggagaagagcaacagccatactcagaaaagcaaccaggagattgattcagctgatagtgag | |
| | tgggagagacgaacagtcgattgccgaagcaataattgtggccatggtattttcacaagaggattgtatga | |
| | taaaagcagtcagaggtgatctgaatttcgtcaatagggcgaatcagcgattgaatcctatgcatcaactt | |
| | ttaagacattttcagaaggatgcgaaagtgctttttcaaaattggggagttgaacctatcgacaatgtgat | |
| | gggaatgattgggatattgccagacatgactccaagcatcgagatgtcaatgagaggagtgagaatcagca | |
| | aaatgggtgtagatgagtactccagcacggagagggtagtggtgagcattgaccgttttttgagaatccgg | |
| | gaccaacgaggaaatgtactactgtctcccgaggaggtcagtgaaacacagggaacagagaaactgacaat | |
| | aacttactcatcgtcaatgatgtgggagattaatggtcctgaatcagtgttggtcaatacctatcaatgga | |
| | tcatcagaaactgggaaactgttaaaattcagtggtcccagaaccctacaatgctatacaataaaatggaa | |
| | tttgaaccatttcagtctttagtacctaaggccattagaggccaatacagtgggtttgtaagaactctgtt | |
| | ccaacaaatgagggatgtgcttgggacatttgataccgcacagataataaaacttcttcccttcgcagccg | |
| | ctccaccaaagcaaagtagaatgcagttctcctcatttactgtgaatgtgaggggatcaggaatgagaata | |
| | cttgtaaggggcaattctcctgtattcaactataacaaggccacgaagagactcacagttctcggaaagga | |
| | tgctggcactttaactgaagacccagatgaaggcacagctggagtggagtccgctgttctgaggggattcc | |
| | tcattctgggcaaagaagacaagagatatgggccagcactaagcatcaatgaactgagcaaccttgcgaaa | |
| | ggagagaaggctaatgtgctaattgggcaaggagacgtggtgttggtaatgaaacggaaacgggactctag | |
| | catacttactgacagccagacagcgaccaaaagaattcggatggccatcaattagtgtcgaatagtttaaa | |
| | aacgaccttgtttctact 3′ | |
| TetON-PB1 | 5′gagtttactccctatcagtgatagagaacgtatgtcgagtttactccctatcagtgatagagaacgatg | SEQ ID NO: 2 |
| (double | tcgagtttactccctatcagtgatagagaacgtatgtcgagtttactccctatcagtgatagagaacgtat | |
| underlined = PB1) | gtcgagtttactccctatcagtgatagagaacgtatgtcgagtttatccctatcagtgatagagaacgtat | |
| | gtcgagtttactccctatcagtgatagagaacgtatgtcgaggtaggcgtgtacggtgggaggcctatata | |
| | agcagagctcgtttagtgaaccgtcagatcgcctggagaattggctagcGCGATCGCCTAGGAATTCGTCG | |
| | ACCTGCAGGTTTAAACggATCagcgaaagcaggcaaaccatttgaatggatgtcaatccgaccttactttt | |
| | cttaaaagtgccagcacaaaatgctataagcacaactttcccttatactggagaccctccttacagccatg | |
| | ggacaggaacaggatacaccatggatactgtcaacaggacacatcagtactcagaaaagggaagatggaca | |
| | acaaacaccgaaactggagcaccgcaactcaacccgattgatgggccactgccagaagacaatgaaccaag | |
| | tggttatgcccaaacagattgtgtattggaagcaatggctttccttgaggaatcccatcctggtatttttg | |
| | aaaactcgtgtattgaaacgatggaggttgttcagcaaacacgagtagacaagctgacacaaggccgacag | |
| | acctatgactggactctaaatagaaaccaacctgctgcaacagcattggccaacacaatagaagtgttcag | |
| | atcaaatggcctcacggccaatgagtctggaaggctcatagacttccttaaggatgtaatggagtcaatga | |
| | aaaaagaagaaatggggatcacaactcattttcagagaaagagacgggtgagagacaatatgactaagaaa | |
| | atgataacacagagaacaataggtaaaaagaagcagagattgaacaaaaggagttatctaattagagcatt | |
| | gaccctgaacacaatgaccaaagatgctgagagagggaagctaaaacggagagcaattgcaaccccaggga | |
| | tgcaaataagggggtttgtatactttgttgagacactggcaaggagtatatgtgagaaacttgaacaatca | |
| | gggttgccagttggaggcaatgagaagaaagcaaagttggcaaatgttgtaaggaagatgatgaccaattc | |
| | tcaggacaccgaactttctttcaccatcactggagataacaccaaatggaacgaaaatcagaatcctcgga | |
| | tgtttttggccatgatcacatatatgacaagaaatcagcccgaatggttcagaaatgttctaagtattgct | |
| | ccaataatgttctcaaacaaaatggcgagactgggaaaagggtatatgtttgagagcaagagtatgaaact | |
| | tagaactcaaatacctgcagaaatgctagcaagcatcgatttgaaatatttcaatgattcaacaagaaaga | |
| | agattgaaaaaatccgaccgctcttaatagaggggactgcatcattgagccctggaatgatgatgggcatg | |
| | ttcaatatgttaagcactgtattaggcgtctccatcctgaatcttggacaaaagagatacaccaagactac | |
| | ttactggtgggatggtcttcaatcctctgacgattttgctctgattgtgaatgcacccaatcatgaaggga | |
| | ttcaagccggagtcgacaggttttatcgaacctgtaagctacttggaatcaatatgagcaagaaaaagtct | |
| | tacataaacagaacaggtacatttgaattcacaagttttttctatcgttatgggtttgttgccaatttcag | |
| | catggagctccccagttttggggtgtctgggatcaacgagtcagcggacatgagtattggagttactgtca | |
| | tcaaaaacaatatgataaacaatgatcttggtccagcaacagctcaaatggcccttcagttgttcatcaaa | |
| | gattacaggtacacgtaccgatgccatagaggtgacacacaaatacaaacccgaagatcatttgaaataaa | |
| | gaaactgtgggagcaaacccgttccaaagctggactgctggtctccgacggaggcccaaatttatacaaca | |
| | ttagaaatctccacattcctgaagtctgcctaaaatgggaattgatggatgaggattaccaggggcgttta | |
| | tgcaacccactgaacccatttgtcagccataaagaaattgaatcaatgaacaatgcagtgatgatgccagc | |
| | acatggtccagccaaaaacatggagtatgatgctgttgcaacaacacactcctggatccccaaaagaaatc | |
| | gatccatcttgaatacaagtcaaagaggagtacttgaagatgaacaaatgtaccaaaggtgctgcaattta | |
| | tttgaaaaattcttccccagcagttcatacagaagaccagtcgggatatccagtatggtggaggctatggt | |
| | ttccagagcccgaattgatgcacggattgatttcgaatctggaaggataaagaaagaagagttcactgaga | |
| | tcatgaagatctgttccaccattgaagagctcagacggcaaaaatagtgaatttagcttgtccttcatgaa | |
| | aaaatgccttgtttctact 3′ | |
| TetON-PA | 5′gagtttactccctatcagtgatagagaacgtatgtcgagtttactccctatcagtgatagagaacgatg | SEQ ID NO: 3 |
| (double | tcgagtttactccctatcagtgatagagaacgtatgtcgagtttactccctatcagtgatagagaacgtat | |
| underlined = PA) | gtcgagtttactccctatcagtgatagagaacgtatgtcgagtttatccctatcagtgatagagaacgtat | |
| | gtcgagtttactccctatcagtgatagagaacgtatgtcgaggtaggcgtgtacggtgggaggcctatata | |
| | agcagagctcgtttagtgaaccgtcagatcgcctggagaattggctagcGCGATCGCCTAGGAATTCGTCG | |
| | ACCTGCAGGTTTAAACggATCagcgaaagcaggtactgatccaaaatggaagattttgtgcgacaatgctt | |
| | caatccgatgattgtcgagcttgcggaaaaaacaatgaaagagtatggggaggacctgaaaatcgaaacaa | |
| | acaaatttgcagcaatatgcactcacttggaagtatgcttcatgtattcagattttcacttcatcaatgag | |
| | caaggcgagtcaataatcgtagaacttggtgatccaaatgcacttttgaagcacagatttgaaataatcga | |
| | gggaagagatcgcacaatggcctggacagtagtaaacagtatttgcaacactacaggggctgagaaaccaa | |
| | agtttctaccagatttgtatgattacaaggagaatagattcatcgaaattggagtaacaaggagagaagtt | |
| | cacatatactatctggaaaaggccaataaaattaaatctgagaaaacacacatccacattttctcgttcac | |
| | tggggaagaaatggccacaaaggcagactacactctcgatgaagaaagcagggctaggatcaaaaccagac | |
| | tattcaccataagacaagaaatggccagcagaggcctctgggattcctttcgtcagtccgagagaggagaa | |
| | gagacaattgaagaaaggtttgaaatcacaggaacaatgcgcaagcttgccgaccaaagtctcccgccgaa | |
| | cttctccagccttgaaaattttagagcctatgtggatggattcgaaccgaacggctacattgagggcaagc | |
| | tgtctcaaatgtccaaagaagtaaatgctagaattgaaccttttttgaaaacaacaccacgaccacttaga | |
| | cttccgaatgggcctccctgttctcagcggtccaaattcctgctgatggatgccttaaaattaagcattga | |
| | ggacccaagtcatgaaggagagggaataccgctatatgatgcaatcaaatgcatgagaacattctttggat | |
| | ggaaggaacccaatgttgttaaaccacacgaaaagggaataaatccaaattatcttctgtcatggaagcaa | |
| | gtactggcagaactgcaggacattgagaatgaggagaaaattccaaagactaaaaatatgaagaaaacaag | |
| | tcagctaaagtgggcacttggtgagaacatggcaccagaaaaggtagactttgacgactgtaaagatgtag | |
| | gtgatttgaagcaatatgatagtgatgaaccagaattgaggtcgctagcaagttggattcagaatgagttt | |
| | aacaaggcatgcgaactgacagattcaagctggatagagctcgatgagattggagaagatgtggctccaat | |
| | tgaacacattgcaagcatgagaaggaattatttcacatcagaggtgtctcactgcagagccacagaataca | |
| | taatgaagggggtgtacatcaatactgccttgcttaatgcatcttgtgcagcaatggatgatttccaatta | |
| | attccaatgataagcaagtgtagaactaaggagggaaggcgaaagaccaacttgtatggtttcatcataaa | |
| | aggaagatcccacttaaggaatgacaccgacgtggtaaactttgtgagcatggagttttctctcactgacc | |
| | caagacttgaaccacataaatgggagaagtactgtgttcttgagataggagatatgcttataagaagtgcc | |
| | ataggccaggtttcaaggcccatgttcttgtatgtgagaacaaatggaacctcaaaaattaaaatgaaatg | |
| | gggaatggagatgaggcgttgcctcctccagtcacttcaacaaattgagagtatgattgaagctgagtcct | |
| | ctgtcaaagagaaagacatgaccaaagagttctttgagaacaaatcagaaacatggcccattggagagtcc | |
| | cccaaaggagtggaggaaagttccattgggaaggtctgcaggactttattagcaaagtcggtattcaacag | |
| | cttgtatgcatctccacaactagaaggattttcagctgaatcaagaaaactgcttcttatcgttcaggctc | |
| | ttagggacaaccttgaacctgggacctttgatcttggggggctatatgaagcaattgaggagtgcctgatt | |
| | aatgatccctgggttttgcttaatgcttcttggttcaactccttccttacacatgcattgagttagttgtg | |
| | gcagtgctactatttgctatccatactgtccaaaaaagtaccttgtttctact 3′ | |

### Example 4B

This Prophetic Example describes construction of a mammalian cell line (also referred to as an iFlu-packaging cell) that expresses a backbone for virus packaging, wherein HA and NA are not integrated into the mammalian cell genome. The cell can, therefore, be provided with different HA and NA antigens (including, for example, by transient transfection).

An iRdRp cell, made as described in Example 1, will be stably transfected with two polynucleotides. The first polynucleotide will encode for vRNAs of PB2, PB1, PA, and NS. The second polynucleotide will encode for both vRNAs and mRNAs of NP and M. The polynucleotides will be operably linked to one ore more RNA polymerase promoters and terminators (for example, Pol I for vRNA and Pol II for mRNA, as shown in FIG. 5B). The polynucleotides will be stably integrated into the genome of the mammalian cell line to create an iFlu-packaging cell.

The iFlu-packaging cell will produce flu virus when transfected with HA and NA under induced conditions. This iFlu-packaging cell may be used to generate different flu virus subtypes when transfected with different subtypes of HA and NA.

### Example 5

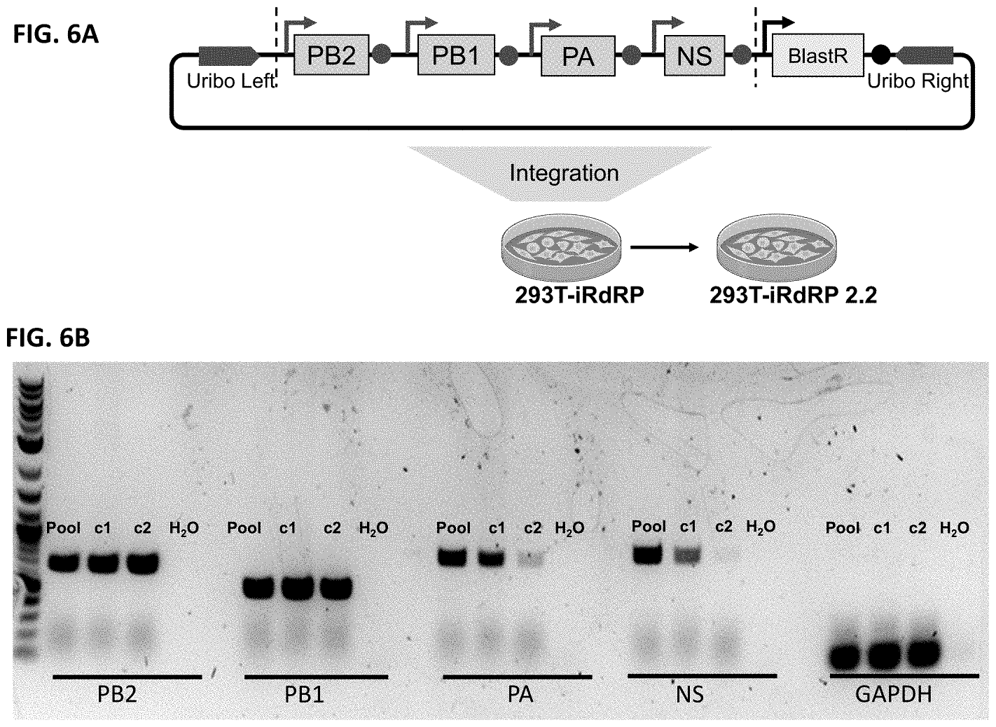

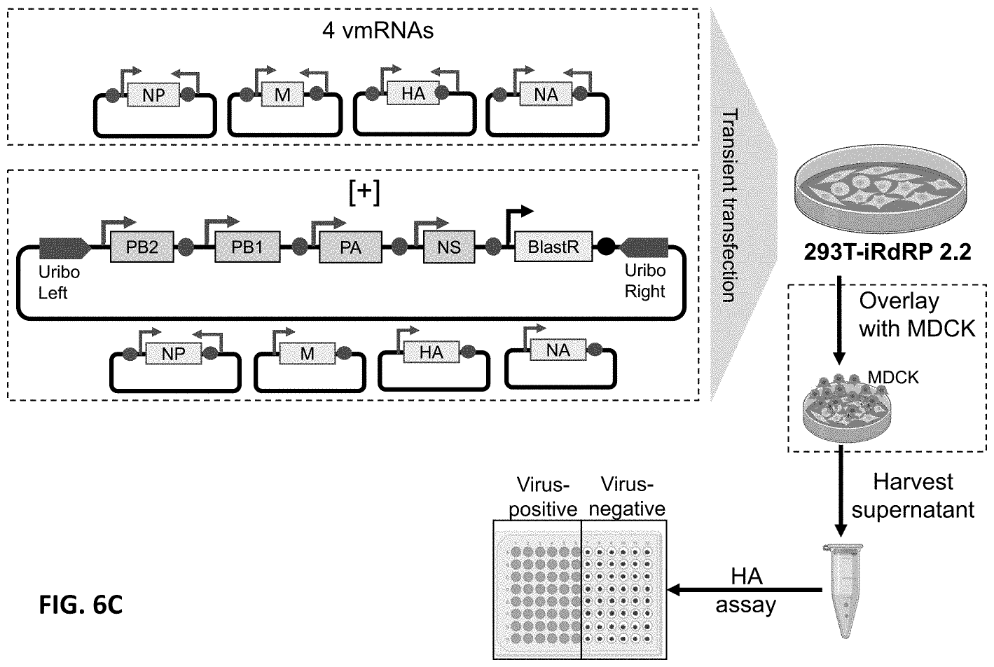

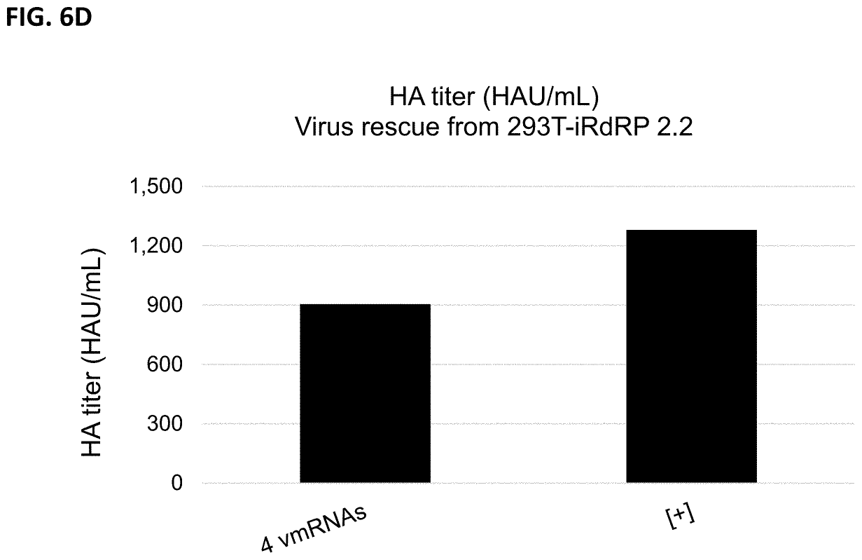

A vector that expresses 4 vRNAs of Influenza A Virus (IAV), named “2.2” was created by inserting polynucleotides encoding vRNAs of PB2, PB1, PA, and NS into ATUM Leap-In transposon backbone vector (ATUM, Newark, Calif.). A schematic of the resulting 2.2 vector is shown in FIG. 6A. This 2.2 vector was used to integrate the DNA sequences that encode the vRNAs of PB2, PB1, PA, and NS into inducible RNA-dependent RNA polymerase (iRdRP)-integrated 293T cells (constructed as described in Example 1) to make 293T-iRdRP 2.2 cells. As further described in Example 1, iRdPP-integrated cells include PB1, PB2, and PA under the control of inducible promoters. The integration was conducted by transfecting vector 2.2 and transposase mRNA (ATUM, Newark, Calif.) into iRdRP cells using Lipofectamine 3000 (Invitrogen, L3000001).

The transfected cell pool (denoted as Pool) was single-cell cloned by dilution to select cell clones that have 4 vRNAs integrated. Clone 1 and clone 2 (denoted as c1 and c2, respectively) represent two single-cell clones that have 4 vRNAs integrated. RNA from Pool, c1, and c2 cells was harvested using RNeasy Mini Kit (Cat. #74504, Qiagen, Hilden, Germany) and used as template to synthesize cDNA using SuperScript™ III First-Strand Synthesis System (Cat. #18080051, Invitrogen, Carlsbad, Calif.) with viral vRNA-specific primers. RT-PCR showed expression of all 4 vRNAs from integrated DNA (FIG. 6B).

293T-iRdRP 2.2 cells were transfected with four plasmids encoding for the missing influenza A PR8 genes (NP, M, NA, HA) (denoted as 4 vmRNAs in FIG. 6C and FIG. 6D) to generate virus, then overlayed with MDCK cells, as described in Neumann et al. (Neumann et al. Proc Natl Acad Sci USA 96, 9345-9350 (1999)) to amplify the signal for hemagglutinin assay to detect virus. For a positive control (denoted as [+] in FIG. 6C and FIG. 6D), the cells were transfected with vector 2.2, pDZ-NP, and 3 plasmids, encoding vRNA of M, HA, or NA. While all vRNAs were supplied transiently in the positive control, the transfection of 4 vmRNAs only supplied the 4 missing genes; vRNAs of PB2, PB1, PA, NS were expressed from integrated DNAs within 293T-iRdRP 2.2 cells. The transfection of 4 vmRNAs only yields virus particle if only 293T-iRdRP 2.2 cells express all 4 full-length vRNAs from the integrated DNAs. A schematic of this transfection is shown in FIG. 6C with the overlay with MDCK cells shown in the dashed box.

The supernatant of the transfected and overlaid cells was harvested 72 hours after overlaying. The supernatant was then used for a hemagglutinin (HA) assay. Results are shown in FIG. 6D. The results indicate that the 293T-iRdRP 2.2 cells can package IAV particles when the missing genes were supplied. These results indicate that 293T-iRdRp 2.2. cells can express both viral mRNAs and vRNAs from integrated DNA sequences. The resulted viral mRNAs and vRNAs can be processed and transported to assemble virus. The results further demonstrate the feasibility of creating an IAV-producing cell line by integrating viral RNA-encoding sequences into DNA genome of the cells.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A mammalian cell line comprising:
a first polynucleotide encoding mRNA of a PB1 subunit of RNA-dependent RNA polymerase (RdRp) of influenza virus, wherein the first polynucleotide is operably linked to a first inducible promoter;
a second polynucleotide encoding mRNA of a PB2 subunit of RdRp of influenza virus, wherein the second polynucleotide is operably linked to a second inducible promoter; and
a third polynucleotide encoding mRNA of a PA subunit of RdRp of influenza virus, wherein the third polynucleotide is operably linked to a third inducible promoter;
wherein the first polynucleotide, the second polynucleotide, and the third polynucleotide are stably integrated into the genome of the mammalian cell.

2. The mammalian cell line of claim 1, the mammalian cell line further comprising:
one or more additional polynucleotides encoding mRNA of HA, NP, NA, M, or NS of influenza virus, or a combination thereof,
wherein the one or more additional polynucleotides are stably integrated into the genome of the mammalian cell.

3. The mammalian cell line of claim 1, the mammalian cell line further comprising:
one or more additional polynucleotides encoding vRNA of PB1, PB2, PA, HA, NP, NA, M, or NS of influenza virus, or a combination thereof,
wherein the one or more additional polynucleotides are stably integrated into the genome of the mammalian cell.

4. The mammalian cell line of claim 3, wherein the one or more additional polynucleotides encoding vRNA comprise DNA.

5. The mammalian cell line of claim 1, wherein the first polynucleotide, the second polynucleotide, and the third polynucleotide comprise DNA and encode positive-sense RNA.

6. A method comprising transiently transfecting the mammalian cell line of claim 1 with one or more additional polynucleotides, the one or more additional polynucleotide encoding vRNA of PB1, PB2, PA, HA, NP, NA, M, or NS of influenza virus, or a combination thereof.

7. A method comprising stably integrating into the mammalian cell line of claim 1 with one or more additional polynucleotides, the one or more additional polynucleotide encoding vRNA of PB1, PB2, PA, HA, NP, NA, M, or NS of influenza virus, or a combination thereof.

8. A method of using the mammalian cell line of claim 1, the method comprising:
generating an infectious influenza virus particle, or
generating an influenza virus protein.

9. The method of claim 8, wherein the influenza virus particle is a replication-deficient influenza virus particle.

10. A method comprising stably integrating into a mammalian cell:
a first polynucleotide encoding mRNA of a PB1 subunit of RNA-dependent RNA polymerase (RdRp) of influenza virus, wherein the first polynucleotide is operably linked to a first inducible promoter;
a second polynucleotide encoding mRNA of a PB2 subunit of RdRp of influenza virus, wherein the second polynucleotide is operably linked to a second inducible promoter; and
a third polynucleotide encoding mRNA of a PA subunit of RdRp of influenza virus, wherein the third polynucleotide is operably linked to a third inducible promoter.

11. The method of claim 10, the method further comprising stably integrating into the mammalian cell:
a fourth polynucleotide encoding mRNA of influenza virus RNA-binding nucleoprotein (NP).

12. The method of claim 10, the method comprising stably integrating into the mammalian cell one or more additional polynucleotides, the one or more additional polynucleotide encoding vRNA of PB1, PB2, PA, HA, NP, NA, M, or NS of influenza virus, or a combination thereof.

13. A mammalian cell line comprising:
a first polynucleotide encoding mRNA of a PB1 subunit of RNA-dependent RNA polymerase (RdRp) of influenza virus, wherein the first polynucleotide is operably linked to a first inducible promoter;
a second polynucleotide encoding mRNA of a PB2 subunit of RdRp of influenza virus, wherein the second polynucleotide is operably linked to a second inducible promoter;
a third polynucleotide encoding mRNA of a PA subunit of RdRp of influenza virus, wherein the third polynucleotide is operably linked to a third inducible promoter;
a fourth polynucleotide encoding mRNA of influenza virus RNA-binding nucleoprotein (NP), wherein the fourth polynucleotide is operably linked to a fourth inducible promoter;
a fifth polynucleotide encoding vRNA of influenza virus NP, wherein the fifth polynucleotide is operably linked to a first RNA polymerase promoter and a first RNA polymerase terminator;
a sixth polynucleotide encoding vRNA of influenza virus non-structural protein (NS), wherein the sixth polynucleotide is operably linked to a second RNA polymerase promoter and a second RNA polymerase terminator;
a seventh polynucleotide encoding vRNA of influenza virus matrix protein (M), wherein the seventh polynucleotide is operably linked to a third RNA polymerase promoter and a third RNA polymerase terminator;
an eighth polynucleotide encoding vRNA of a PB1 subunit of RdRp of influenza, wherein the eighth polynucleotide is operably linked to a fourth RNA polymerase promoter and a fourth RNA polymerase terminator;
a ninth polynucleotide encoding vRNA of a PB2 subunit of RdRp of influenza, wherein the ninth polynucleotide is operably linked to a fifth RNA polymerase promoter and a fifth RNA polymerase terminator; and
a tenth polynucleotide encoding vRNA of a PA subunit of RdRp of influenza, wherein the tenth polynucleotide is operably linked to a sixth RNA polymerase promoter and a sixth RNA polymerase terminator.

14. The mammalian cell line of claim 13 further comprising:
an eleventh polynucleotide encoding vRNA of influenza virus hemagglutinin (HA), wherein the eleventh polynucleotide is operably linked to a seventh RNA polymerase promoter and a seventh RNA polymerase terminator; or
a twelfth polynucleotide encoding vRNA of influenza virus neuraminidase (NA), wherein the twelfth polynucleotide is operably linked to an eighth RNA polymerase promoter and an eighth polymerase terminator; or
both.

15. The mammalian cell line of claim 14, further comprising:
an eleventh polynucleotide encoding an influenza virus genome segment wherein the eleventh polynucleotide is operably linked to a seventh RNA polymerase promoter and a seventh RNA polymerase terminator,
wherein the influenza virus genome segment comprises 3′ and 5′ packaging elements of vRNA of influenza virus hemagglutinin (HA), and
wherein the influenza virus genome segment comprises a coding sequence of influenza HA vRNA, and further wherein the influenza virus genome segment comprises a defect in the coding sequence of influenza HA vRNA.

16. The mammalian cell line of claim 13, wherein:
the first polynucleotide further comprises the eighth polynucleotide;
the second polynucleotide further comprises the ninth polynucleotide;
the third polynucleotide further comprises the tenth polynucleotide; and
a fourth polynucleotide further comprises the fifth polynucleotide.

17. The mammalian cell line of claim 13, wherein the first polynucleotide, the second polynucleotide, the third polynucleotide, the fourth polynucleotide, the fifth polynucleotide, the sixth polynucleotide, the seventh polynucleotide, the eighth polynucleotide, the ninth polynucleotide, and the tenth polynucleotide are stably integrated into the genome of the mammalian cell line.

18. A method of using the mammalian cell line of claim 13 the method comprising:
generating an infectious influenza virus particle, wherein the influenza virus particle is optionally a replication-deficient influenza virus particle, and/or
generating an influenza virus protein.

19. The method of claim 18, the method comprising exposing the mammalian cell line to inducers of the first inducible promoter, the second inducible promoter, the third inducible promoter, and the fourth inducible promoter.

20. A method of making the mammalian cell line of claim 13.


SEQUENCE LISTING (3 sequences)

SEQ ID NO: 1 - DNA, 2715 bp, artificial
  inducible RdRP construct
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattggctagcgcgatcgcctaggaattcgtcgacctgcaggtttaaacggatcagcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgcaggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcagcgattgaatcctatgcatcaacttttaagacattttcagaaggatgcgaaagtgctttttcaaaattggggagttgaacctatcgacaatgtgatgggaatgattgggatattgccagacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 2 - DNA, 2715 bp, artificial
  inducible RdRP construct
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattggctagcgcgatcgcctaggaattcgtcgacctgcaggtttaaacggatcagcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgacaagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 3 - DNA, 2607 bp, artificial
  inducible RdRP construct
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattggctagcgcgatcgcctaggaattcgtcgacctgcaggtttaaacggatcagcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgctagcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact